(19) United States
(12) Patent Application Publication
(10) Pub. No.: US 2022/0308044 A1
(43) Pub. Date: Sep. 29, 2022

(54) NOVEL LIVE-CELL ASSAY FOR NEURONAL ACTIVITY

(71) Applicant: UNIVERSITY OF UTAH RESEARCH FOUNDATION, Salt Lake City, UT (US)

(72) Inventors: SungJin Park, Salt Lake City, UT (US); Ana Santos, Salt Lake City, UT (US); Kevin Huang, Salt Lake City, UT (US)

(21) Appl. No.: 17/616,111

(22) PCT Filed: Jun. 19, 2020
(86) PCT No.: PCT/US2020/038716

§ 371 Date: Dec. 2, 2021

Related U.S. Application Data
(60) Provisional application No. 62/864,612, filed on Jun. 21, 2019.

(51) Int. Cl. G01N 33/50; C12N 15/86; C12N 9/02

(52) U.S. Cl. CPC G01N 33/5058; C12N 15/86; C12N 9/0069; C12N 2740/15043

(57) ABSTRACT

Disclosed herein are neuronal cell activity reporter systems including a Secreted Neuronal Activity Reporter (SNAR) construct and a control construct. The SNAR construct includes four tandem repeats of a core domain of the Synaptic Activity Response Element (SARE) of Arc/Arg3.1, a polynucleotide comprising the Arc minimal promoter, and a polynucleotide encoding a first secreted reporter protein. The control construct includes a constitutive promoter and a polynucleotide encoding a second secreted reporter protein. Further provided are methods of monitoring neuronal activity in a cell. The methods may include administering to a cell the neuronal cell activity reporter system, contacting with a substrate, and measuring a signal.

### CROSS-REFERENCE TO RELATED APPLICATION(S)

This application claims priority to U.S. Provisional Patent Application No. 62/864,612, filed Jun. 21, 2019, which is incorporated herein by reference in its entirety.

### STATEMENT REGARDING FEDERALLY SPONSORED RESEARCH

This invention was made with government support under grant no. NS102444 awarded by the National Institutes of Health. The government has certain rights in the invention.

### INCORPORATION-BY-REFERENCE OF MATERIAL SUBMITTED ELECTRONICALLY

Incorporated herein by reference in its entirety is a computer-readable nucleotide/amino acid sequence listing submitted concurrently herewith and identified as follows: One 11,669 Byte ASCII (Text) file named “026389-9258-WO01-SEQ-LIST-06-19-20_ST25.txt,” created on Jun. 18, 2020.

### FIELD

This disclosure relates to compositions and methods for monitoring the activity of live neurons.

### INTRODUCTION

Proper synapse development and function are factors in the formation of neuronal circuits and information processing. Misregulation of synaptic development and wiring can cause many forms of neurodevelopmental and neuropsychiatric disorders including intellectual disability, epilepsy, autism spectrum disorders (ASD), and schizophrenia. Mechanisms of synapse formation and maturation have been extensively studied. Many synaptic molecules have been identified and characterized, which largely include synaptic adhesion molecules and scaffolding proteins. However, despite advances in knowledge of synaptic molecules in both normal and disease conditions, intervening therapeutics remain limited, partly because cell adhesion/scaffolding proteins are difficult drug targets. Moreover, due to homeostatic mechanisms, neurons are able to regulate neuronal activity levels in response to prolonged external signals, making long-term effects of drug treatment often different from the initial response. This can be problematic since it is more challenging to distinguish acute effects from long-term effects during drug screens.

Efficient live cell assays that allow for quantification of changes in neuronal activity over time would be helpful to identify and characterize small molecules that modulate synapse development and function. Current methods to study synapse formation and function largely rely on immunostaining of fixed neurons and electrophysiological analyses of individual neurons. These methods provide spatial resolution, molecular composition, and detailed mechanistic insights on synaptic transmission of individual neurons. However, they are not ideal to directly compare drug effects on the same population of neurons or to longitudinally monitor synapse development due to a single time point being obtained for analysis. Furthermore, due to the heterogeneity of cultured neurons, a large amount of data from blind experiments may be needed for statistical analysis, making it impractical for use in medium- and high-throughput screens.

Alternatively, non-invasive methods have been developed to monitor the development of neuronal activity, including live cell imaging and multielectrode arrays (MEA). Optical approaches include the use of genetically encoded fluorescent sensors, including calcium indicators, neurotransmitter sensors, and voltage indicators. Although these reporters may be used for live cell imaging of network activity during a short period of time, fluorescence-based methods are prone to photobleaching and other caveats such as phototoxicity and imprecise quantification. Long-term monitoring of multiple samples over days may require a microscope equipped with a sophisticated tracking device, which limits its application to large-scale analyses. MEA has been used for longitudinal monitoring of population activity. Although MEA non-invasively monitors network activity and is useful to identify drugs that affect overall population activity, the high cost of a disposable culture plate and non-selectivity may limit its application to large-scale screens for specific types of neurons. There is a need for efficient live cell assays that allow for quantification of changes in neuronal activity over time.

### SUMMARY

In an aspect, the disclosure relates to a Secreted Neuronal Activity Reporter (SNAR) construct. The SNAR construct may include four tandem repeats of a core domain of the Synaptic Activity Response Element (SARE) of Arc/Arg3.1; a polynucleotide comprising the Arc minimal promoter; and a polynucleotide encoding a first secreted reporter protein. In some embodiments, the core domain of the SARE of Arc/Arg3.1 comprises a polynucleotide of SEQ ID NO: 2. In some embodiments, the Arc minimal promoter comprises a polynucleotide of SEQ ID NO: 3. In some embodiments, the first secreted reporter protein emits a light signal upon contact with a substrate. In some embodiments, the substrate comprises coelenterazine. In some embodiments, the first secreted reporter protein comprises Gaussia luciferase. In some embodiments, the Gaussia luciferase comprises a polypeptide of SEQ ID NO: 7. In some embodiments, the SNAR construct further includes a loxP site upstream of the polynucleotide encoding a first secreted reporter protein, and a lox2272 site downstream of the polynucleotide encoding a first secreted reporter protein. In some embodiments, the SNAR construct further includes a loxP site downstream of the polynucleotide encoding a first secreted reporter protein, and a lox2272 site upstream of the polynucleotide encoding a first secreted reporter protein. In some embodiments, the loxP site comprises a polynucleotide of SEQ ID NO: 4, and the lox2272 site comprises a polynucleotide of SEQ ID NO: 5.

In a further aspect, the disclosure relates to a neuronal cell activity reporter system including (a) the SNAR construct as detailed herein; and (b) a control construct comprising: a polynucleotide comprising a constitutive promoter; and a polynucleotide encoding a second secreted reporter protein.

Another aspect of the disclosure provides a method of monitoring neuronal activity in a test cell. The method may include administering to the test cell the SNAR construct as detailed herein; contacting the first secreted reporter protein with a substrate, wherein the substrate reacts with the first secreted reporter protein to generate a first signal (CTZₛₐₘₚₗₑ); measuring the first signal; and determining the neuronal activity in the test cell based on the first signal. In some embodiments, the substrate comprises coelenterazine. In some embodiments, the first secreted reporter protein is exported out of the test cell to a culture medium. In some embodiments, the first secreted reporter protein is contacted with the substrate by adding the substrate to a sample of the culture medium. In some embodiments, the first signal is measured at two different time points, and the neuronal activity in the test cell at the two different time points are compared. In some embodiments, the neuronal activity in the test cell is monitored by measuring the first signal at a plurality of different time points.

Another aspect of the disclosure provides a method of monitoring neuronal activity in a test cell, wherein the method may include (a) administering to the test cell the SNAR construct as detailed herein, and a control construct, the control construct comprising: a polynucleotide comprising a constitutive promoter; and a polynucleotide encoding a second secreted reporter protein; (b) contacting the first secreted reporter protein and the second secreted reporter protein in the test cell with a first substrate, wherein the first substrate reacts with the first secreted reporter protein and the second secreted reporter protein to generate a first signal (CTZₛₐₘₚₗₑ); (c) measuring the first signal; (d) contacting the first secreted reporter protein and the second secreted reporter protein in the test cell with a second substrate, wherein the second substrate reacts with the second secreted reporter protein to generate a second signal (FMZₛₐₘₚₗₑ); (e) measuring the second signal; (f) administering to a control cell the control construct of step (a); (g) contacting the second secreted reporter protein in the control cell with the first substrate, wherein the first substrate reacts with the second secreted reporter protein to generate a third signal (CTZ_{sNluc}); (h) measuring the third signal; (i) contacting the second secreted reporter protein in the control cell with the second substrate, wherein the second substrate reacts with the second secreted reporter protein to generate a fourth signal (FMZ_{sNluc}); (j) measuring the fourth signal; (k) determining a control ratio by dividing the third signal by the fourth signal (CTZ_{sNluc}/FMZ_{sNluc}); and (l) determining the neuronal activity in the test cell based on the contribution of the first secreted reporter protein to the first signal with the control ratio. In some embodiments, the contribution of the first secreted reporter protein to the first signal is calculated by subtracting from the first signal the product of the control ratio and the second signal (first signal−[(third signal/fourth signal]×second signal]=CTZₛₐₘₚₗₑ−[(CTZ_{sNluc}/FMZ_{cNluc})×FMZₛₐₘₚₗₑ]).

In some embodiments, the constitutive promoter comprises a human PGK promoter. In some embodiments, the human PGK promoter comprises a polynucleotide of SEQ ID NO: 6. In some embodiments, the second secreted reporter protein emits a signal upon contact with a substrate, the signal being distinct from the signal emitted by the first secreted reporter protein upon contact with a substrate. In some embodiments, the second secreted reporter protein emits a signal upon contact with furimazine, coelenterazine, or a combination thereof. In some embodiments, the first substrate comprises coelenterazine. In some embodiments, the second substrate comprises furimazine. In some embodiments, the second secreted reporter protein comprises a nanoluciferase comprising an N-terminal secretion signal peptide. In some embodiments, the nanoluciferase comprising an N-terminal secretion signal peptide comprises a polypeptide of SEQ ID NO: 9. In some embodiments, the control construct further comprises a loxP site upstream of the polynucleotide encoding a second secreted reporter protein, and a lox2272 site downstream of the polynucleotide encoding a second secreted reporter protein. In some embodiments, the control construct further comprises a loxP site downstream of the polynucleotide encoding a second secreted reporter protein, and a lox2272 site upstream of the polynucleotide encoding a second secreted reporter protein. In some embodiments, the loxP site comprises a polynucleotide sequence of SEQ ID NO: 4, and the lox2272 site comprises a polynucleotide sequence of SEQ ID NO: 5. In some embodiments, the first secreted reporter protein and the second secreted reporter protein are exported out of the test cell to a culture medium. In some embodiments, the first secreted reporter protein and the second secreted reporter protein are contacted with the first substrate by adding the first substrate to a sample of the culture medium. In some embodiments, the first secreted reporter protein and the second secreted reporter protein are contacted with the second substrate by adding the second substrate to a sample of the culture medium. In some embodiments, the first signal and the second signal are measured at two different time points, and the neuronal activity in the test cell at the two different time points are compared. In some embodiments, the neuronal activity in the test cell is monitored by measuring the first signal and the second signal at a plurality of different time points. In some embodiments, the method further comprises contacting the test cell with a Cre recombinase. In some embodiments, the test cell is a live cell. In some embodiments, the method further includes contacting the test cell with a modulator of synaptic signaling. In some embodiments, the SNAR construct is an adeno-associated virus (AAV) or a lentivirus.

The disclosure provides for other aspects and embodiments that will be apparent in light of the following detailed description and accompanying figures.

### BRIEF DESCRIPTION OF THE DRAWINGS

FIG. 1A-FIG. 1D. AARE Reporter reflects neuronal activity and endogenous Arc levels. (FIG. 1A) Diagram of SNAR constructs and experimental paradigm. The activity dependent reporter (Secreted Neuronal Activity Reporter, SNAR) consists of the core domain of the SARE sequence (cSARE) and the Arc/Arg3.1 minimal promoter coupled with Gaussia Luciferase (Gluc). A control construct consists of a constitutive promoter (hPGK) followed by secreted Nanoluciferase (sNluc). Samples can be collected at many times points as needed (green bars). (FIG. 1B) Gluc and Nluc activity can be measured reliably from mixed samples. 293T cells were transfected with either pCAG_Gluc or pCAG_sNluc. Various combinations of media were prepared (input ratio) and the activity of each luciferase measured (calculated ratio, n=3). (FIG. 1C) SNAR is neuronal activity dependent. WT neurons were treated with either an inhibitor cocktail (2 μM TTX, 200 μM AP5, 8 μM CNQX), picrotoxin (50 μM PTX), or vehicle/DMSO for 46-48 hours total. Quantification of change in SNAR activity (right) was normalized to 16 hours to avoid detection of pre-existing transcripts (n=4, bonferroni p-values post ANOVA). (FIG. 1D) Washout of inhibitors from FIG. 1C shows a rapid increase in luciferase activity (normalized to activity at t=0 min and Nluc).

FIG. 2A-FIG. 2C. Longitudinal measurement of neuronal activity. (FIG. 2A) Hippocampal neurons were cultured and provided with either neuronal media conditioned in astrocytes (ACM) or unconditioned media (no ACM) with every half media change every three days starting from DIV7. (FIG. 2B) SNAR activity increased over neuronal maturation. Luciferase accumulation is normalized to before treatment (mean+/−SEM, n=3). (FIG. 2C) Quantification of the daily accumulation in FIG. 2B.

FIG. 3A-FIG. 3C. Pharmacological and kinetic analysis of SNAR Activity. (FIG. 3A) Treatment of WT neurons with various inhibitors bidirectionally regulated the SNAR activity. Blocking NMDAR-mediated transmission by AP5 reduced the SNAR activity, while blocking AMPAR-mediated transmission by CNQX treatment increased it (mean+/−SEM***Bonferroni p<0.001). (FIG. 3B) Kinetic analysis showed that CNQX treatment induced the delayed increase in the SNAR activity 16 hours after the treatment. Data was normalized to t=0 h (mean+/−SEM, n=7 for Ctrl, 8 for CNQX, Student's t-test at 16 h p=0.17 and at 40 h p=0.012). (FIG. 3C) Inhibition of ERK signaling pathway by U0126 or of L-type calcium channels by Nifedipine (10 μM) reduced the SNAR activity (mean+/−SEM, Bonferroni p<0.001).

FIG. 4A-FIG. 4B. Bidirectional modulation of SNAR. Treatment with anti-seizure drugs reduced SNAR, while treatment with a neurotrophic factor induced SNAR. (FIG. 4A) Treatment of WT neurons with anti-seizure drugs. PHT, phenytoin (80 μM) and CBZ, carbamazepine (50 μM) (mean+/−SEM, n=4). (FIG. 4B) BDNF treatment (50 ng/mL) induced SNAR expression both at 16 hours and 40 hours after the treatment (mean+/−SEM, n=4, Student's t-test, 16 h p<0.01, 40 h p<0.001).

FIG. 5A-FIG. 5D. Cell-type specificity of SNAR. (FIG. 5A) SNAR was expressed mostly in CamKII-positive neurons. (FIG. 5B) Although the majority of inhibitory neurons (GAD67+) did not express SNAR (arrowheads), a small subpopulation (˜10%) did express Gluc (arrows). (FIG. 5C) Quantification of FIG. 5A and FIG. 5B. (FIG. 5D) SNAR was specifically expressed in neurons (MAP2-positive cells) and not astrocytes (GFAP-positive cells). Scale bar 100 μm.

FIG. 6A-FIG. 6C. Expression of SNAR in a subpopulation of neurons. (FIG. 6A) Diagram of Cre-dependent constructs used. We used a double-floxed inverted open reading frame cassette (D10). (FIG. 6B) Neurons transduced with the floxed constructs depicted in FIG. 6A expressed little to no luciferase, while neurons transduced with both CamKII-Cre and SNAR showed robust Cre recombination and high expression of luciferase. (FIG. 6C) SNAR expression remained largely mediated by NMDA receptors in CamKII neurons (mean+/−SEM, n=4, Student's t-test p<0.001).

FIG. 7A-FIG. 7C. The SNAR construct. (FIG. 7A) Core SARE sequence (cSARE) used to build SNAR. Boxes indicate conserved sequences previously shown to correspond to transcription factor binding sites. (FIG. 7B) The SNAR consists of the core SARE sequence repeated four times (4×) followed by the Arc minimal promoter and the Gaussia luciferase (Gluc) coding sequence. (FIG. 7C) The control construct for luciferase assays consists of the hPGK promoter followed by a secreted form of Nanoluciferase (Nluc). Nluc was converted into a secreted protein by introducing the Ig-kappa signal peptide (SP) preceding its coding sequence.

FIG. 8A-FIG. 8E. Validation of Dual Luciferase System using Gluc and Nluc. (FIG. 8A) Gluc and Nluc linearly accumulate in the media over time in naïve conditions. 293T cells were transfected with equal amounts of a plasmid encoding pCAG_Gluc (left) or pCAG_Nluc (right). The next day, media was sampled every hour for 6 hours and luciferase activity assayed. (FIG. 8B) Kinetics of each luciferase are not affected in mixed samples from 293T cells. Kinetic plots are shown for FMZ (left) and CTZ (right) luciferase reactions. (FIG. 8C) Linear increase in luciferase activity correlates with concentration of sNluc both in CTZ (left) and FMZ reactions (right). (FIG. 8D) Formula to calculate Gluc from mixed samples. (FIG. 8E) Stability of each luciferase in vitro. Neurons were infected at DIV1 and luciferase allowed to accumulate in the media for 7 days, at which point it was transferred to an uninfected neuron culture of the same age (n=5). The activity of each luciferase was monitored every day until DIV14.

FIG. 9A-FIG. 9B. The SNAR assay requires a minimal volume of sample. (FIG. 9A) SNAR activity from several dilutions of a sample. (FIG. 9B) Gluc kinetics from the most diluted sample in FIG. 9A, 1:100 dilution.

### DETAILED DESCRIPTION

Described herein are live cell assays that enable the quantification of changes in neuronal activity in live neurons multiple times by combining an activity-dependent driver, based on Arc gene regulatory elements, and a secreted reporter protein. Longitudinal monitoring of the accumulated secreted reporter protein in the medium may reveal the developmental dynamics of neuronal activity in different culture conditions. Direct comparison of changes in neuronal activity within the same population of neurons upon pharmacological manipulation may improve the consistency of assays by reducing variation among cultures. Because the reporter is amenable to repeated measurements, kinetic analyses can be performed, which may facilitate the distinction of short and long-term effects of pharmacological manipulations. Conditional expression of the reporter by using Cre recombinase may be used and may allow for selective monitoring of neuronal activity in a sub-population of neurons in heterogeneous cultures. The simple, quantitative, and selective activity reporter assay may be used to study the development of neuronal activity in normal and disease conditions and to identify small molecules/protein factors that selectively modulate the neuronal activity of specific populations of neurons.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “administration” or “administering,” as used herein, refers to providing, contacting, and/or delivery of a compound, vector, or agent, etc., by any appropriate route to achieve the desired effect. These compounds or agents may be administered to a subject in numerous ways including, but not limited to, orally, ocularly, nasally, intravenously, topically, as aerosols, suppository, etc. and may be used in combination.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “antagonist” or “inhibitor” refers to a substance that blocks (e.g., reduces or prevents) a biological activity. An inhibitor may inhibit an activity directly or indirectly.

As used herein, the term “agonist” refers to a substance that triggers (e.g., initiates or promotes), partially or fully enhances, stimulates, or activates one or more biological activities. An agonist may mimic the action of a naturally occurring substance. Whereas an agonist causes an action, an antagonist blocks the action of the agonist.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be a subject, or a sample therefrom, whose disease state is known. The subject, or sample therefrom, may be healthy, diseased, diseased prior to treatment, diseased during treatment, diseased after treatment, or healthy after treatment, or a combination thereof. The term “normal subject” as used herein means a healthy subject, i.e. a subject having no clinical signs or symptoms of disease. The normal subject is clinically evaluated for otherwise undetected signs or symptoms of disease, which evaluation may include routine physical examination and/or laboratory testing. In some embodiments, the control is a healthy control. In some embodiments, the control comprises neurodegenerative disease. In some embodiments, the control has a wild-type phenotype and/or genotype.

As used herein, the term “cloning” refers to the process of ligating a polynucleotide into a vector and transferring it into an appropriate host cell for duplication during propagation of the host.

The term “effective amount,” as used herein, refers to a dosage effective for eliciting a desired effect. This term as used herein may also refer to an amount effective at bringing about a desired in vivo effect in a subject, such as in an animal, preferably, a human, such as treatment of a disease.

The term “host cell” is a cell that is susceptible to transformation, transfection, transduction, conjugation, and the like with a polynucleotide construct or expression vector. Host cells can be prokaryotic. Host cells can be eukaryotic. Host cells can be derived from animals, plants, bacteria, yeast, fungi, insects, animals, protozoans, etc.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

Polynucleotides are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a polynucleotide sequence, even if internal to a larger oligonucleotide, also may be said to have 5′ and 3′ ends. In either a linear or circular polynucleotide, discrete elements are referred to as being “upstream” or 5′ of the “downstream” or 3′ elements. This terminology reflects the fact that transcription proceeds in a 5′ to 3′ fashion along the polynucleotide strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “gene” means the polynucleotide sequence comprising the coding region of a gene, e.g., a structural gene, and the including sequences located adjacent to the coding region on both the 5′ and 3′ ends for a distance of, for example, about 1 kb on either end such that the gene corresponds to the length of the full-length mRNA. The sequences which are located 5′ or upstream of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA, for example, heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, post-transcriptional cleavage and polyadenylation.

As used herein, an oligonucleotide or polynucleotide “having a nucleotide sequence encoding a gene” means a polynucleotide sequence comprising the coding region of a gene, or in other words, the nucleic acid sequence which encodes a gene product. The coding region may be present in either a cDNA, genomic DNA, or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the vector may contain endogenous enhancers, promoters, splice junctions, intervening sequences, polyadenylation signals, etc., or a combination of both endogenous and exogenous control elements.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Recombinant” when used with reference, e.g., to a cell, or polynucleotide, protein, or vector, indicates that the cell, nucleic acid, protein, or vector, has been modified by the introduction of a heterologous nucleic acid or protein or the alteration of a native polynucleotide or protein, or that the cell is derived from a cell so modified. Thus, for example, recombinant cells express genes that are not found within the native (non-recombinant) form of the cell or express native genes that are otherwise abnormally expressed, under expressed, or not expressed at all. For example, the term “recombinant DNA molecule” as used herein refers to a DNA molecule which is comprised of segments of DNA joined together by means of molecular biological techniques. The term “recombinant protein” or “recombinant polypeptide” as used herein refers to a protein molecule which is expressed from a recombinant DNA molecule or recombinant polynucleotide.

The term “native protein” as used herein to indicate that a protein does not contain amino acid residues encoded by vector sequences; the native protein contains only those amino acids found in the protein as it occurs in nature. A native protein may be produced by recombinant means or may be isolated from a naturally occurring source.

An “open reading frame” includes at least 3 consecutive codons which are not stop codons. The term “codon” as used herein refers to any group of three consecutive nucleotide bases in a given messenger RNA molecule, or coding strand of DNA or polynucleotide that specifies a particular amino acid, a starting signal, or a stopping signal for translation. The term codon also refers to base triplets in a DNA strand.

The terms “in operable combination,” “in operable order,” and “operably linked” as used herein, refer to a functional combination between a promoter region and a nucleotide sequence such that the transcription of the nucleotide sequence is controlled and regulated by the promoter region. Techniques for operatively linking a promoter region to a nucleotide sequence are known in the art. The term may refer to the linkage of polynucleotide sequences in such a manner that a polynucleotide molecule capable of directing the transcription of a given gene and/or the synthesis of a desired protein molecule is produced. The term also refers to the linkage of amino acid sequences in such a manner so that a functional protein is produced.

As used herein, the term “restriction endonuclease” or “restriction enzyme” refers to a member or members of a classification of catalytic molecules that bind a cognate sequence of a polynucleotide and cleave the polynucleotide at a precise location within that sequence. Restriction endonuclease may be bacterial enzymes. Restriction endonuclease may cut double-stranded DNA at or near a specific nucleotide sequence.

As used herein, “recognition site” or “restriction site” refers to a sequence of specific bases or nucleotides that is recognized by a restriction enzyme if the sequence is present in double-stranded DNA; or, if the sequence is present in single-stranded RNA, the sequence of specific bases or nucleotides that would be recognized by a restriction enzyme if the RNA was reverse transcribed into cDNA and the cDNA employed as a template with a DNA polymerase to generate a double-stranded DNA; or, if the sequence is present in single-stranded DNA, the sequence of specific bases or nucleotides that would be recognized by a restriction enzyme if the single-stranded DNA was employed as a template with a DNA polymerase to generate a double-stranded DNA; or, if the sequence is present in double-stranded RNA, the sequence of specific bases or nucleotides that would be recognized by a restriction enzyme if either strand of RNA was reverse transcribed into cDNA and the cDNA employed as a template with a DNA polymerase to generate a double-stranded DNA. The term “unique restriction enzyme site” or “unique recognition site” indicates that the recognition sequence for a given restriction enzyme appears once within a polynucleotide.

As used herein, the term “regulatory element” refers to a genetic element which controls some aspect of the expression of polynucleotide sequences. A regulatory element may also be referred to as a transcription element. A “promoter” is a regulatory element that facilitates the initiation of transcription of an operably linked coding region. A promoter is the regulatory DNA region which controls transcription or expression of a gene and which can be located adjacent to or overlapping a nucleotide or region of nucleotides at which RNA transcription is initiated. A promoter contains specific DNA sequences which bind protein factors, often referred to as transcription factors, which facilitate binding of RNA polymerase to the DNA leading to gene transcription. Other regulatory elements may include splicing signals, polyadenylation signals, termination signals, and the like. The term “constitutive promoter” refers to a promoter active in all or most tissues of an organism at all or most developing stages. Transcriptional control signals in eukaryotes include “promoter” and “enhancer” elements. Promoters and enhancers include short arrays of polynucleotide sequences that interact specifically with cellular proteins involved in transcription (Maniatis et al., Science, 236: 1237 (1987), incorporated herein by reference). Conventional promoter and enhancer elements have been isolated from a variety of eukaryotic sources such as, for example, genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss et al., Trends Biochem. Sci. 1986, 11, 287 and Maniatis et al., supra (1987)). For example, the SV40 early gene enhancer is very active in a wide variety of cell types from many mammalian species and has been widely used for the expression of proteins in mammalian cells (Dijkema et al. EMBO J. 1985, 4, 761). Two other examples of promoter/enhancer elements active in a broad range of mammalian cell types are those from the human elongation factor 10 gene (Uetsuki et al. J. Biol. Chem. 1989, 264, 5791; Kim et al. Gene 1990, 91, 217; Mizushima et al. Nuc. Acids. Res. 1990, 18, 5322) and the long terminal repeats of the Rous sarcoma virus (Gorman et al. Proc. Natl. Acad. Sci. USA 1982, 79, 6777) and the human cytomegalovirus (Boshart et al. Cell 1985, 41, 521). As used herein, the term “promoter/enhancer” denotes a segment of a polynucleotide that contains sequences capable of providing both promoter and enhancer functions (i.e., the functions provided by a promoter element and an enhancer element). For example, the long terminal repeats of retroviruses contain both promoter and enhancer functions. The regulatory element may be “endogenous” or “exogenous” or “heterologous.” An “endogenous” regulatory element is one which is naturally linked with a given gene in the genome. An “exogenous” or “heterologous” regulatory element is one which is placed in juxtaposition to a gene by means of genetic manipulation (i.e., molecular biological techniques) such that transcription of that gene is directed by the linked regulatory element.

“Replication origins” are unique polynucleotide segments that contain multiple short repeated sequences that are recognized by multimeric origin-binding proteins and which play a key role in assembling DNA replication enzymes at the origin site.

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York (1989) pp. 16.7-16.8). An example of a splice donor and acceptor site is the splice junction from the 16S RNA of SV40.

As used herein, the term “purified” or “to purify” or “isolate” refers to the removal of contaminants from a sample.

As used herein the term “portion” when in reference to a protein or polynucleotide (as in “a portion of a given protein”) refers to fragments of that protein or polynucleotide. The protein fragments may range in size from two or more amino acid residues to the entire amino acid sequence minus one amino acid. Polynucleotide fragments may range in size from two or more nucleotides to the entire polynucleotide sequence minus one nucleotide.

The term “specificity” as used herein refers to the number of true negatives divided by the number of true negatives plus the number of false positives, where specificity (“spec”) may be within the range of 0<spec<1. Hence, a method that has both sensitivity and specificity equaling one, or 100%, is preferred.

“Sample” or “test sample” as used herein can mean any sample in which the presence and/or level of an activity, a biomarker, target, agent, vector, or molecule, etc., is to be detected or determined. Samples may include liquids, solutions, emulsions, mixtures, or suspensions. Samples may include a medical sample. Samples may include any biological fluid or tissue, such as blood, whole blood, fractions of blood such as plasma and serum, peripheral blood mononuclear cells (PBMCs), muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Samples may be obtained before treatment, before diagnosis, during treatment, after treatment, or after diagnosis, or a combination thereof.

As used herein, the term “selectable marker” or “selectable marker gene” refers to the use of a gene which encodes an enzymatic activity that confers the ability to grow in medium lacking what would otherwise be an essential nutrient (e.g., the TRPI gene in yeast cells), and/or confer upon the cell resistance to an antibiotic or drug in which the selectable marker is expressed. Selection markers may provide a means to select for or against growth of cells which have been successfully transformed with a vector containing the selection marker sequence and express the marker. A selectable marker may be used to confer a particular phenotype upon a host cell. When a host cell must express a selectable marker to grow in selective medium, the marker is said to be a positive selectable marker (e.g., drug or antibiotic resistance genes which confer the ability to grow in the presence of the appropriate antibiotic, or enable cells to detoxify an exogenously added drug that would otherwise kill the cell). Another example of a positive selection marker is a an auxotrophic marker, which allows cells to synthesize an essential component (usually an amino acid) while grown in media which lacks that essential component. Selectable auxotrophic gene sequences include, for example, hisD, which allows growth in histidine free media in the presence of histidinol. Selectable markers can also be used to select against host cells containing a particular gene (e.g., the sacB gene which, if expressed, kills the bacterial host cells grown in medium containing 5% sucrose); selectable markers used in this manner are referred to as negative selectable markers or counter-selectable markers. In some embodiments, selectable markers include resistance genes such as antibiotic resistance genes.

“Subject” as used herein can mean an organism that wants or is in need of the herein described compounds or methods. The subject may be a human or a non-human animal. The subject may be a microorganism. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant. The subject may be male. The subject may be female.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

The terms “transformation” and “transfection” as used herein refer to the introduction of foreign DNA or polynucleotide into prokaryotic or eukaryotic cells. Transformation of prokaryotic cells may be accomplished by a variety of means known to the art including, for example, the treatment of host cells with CaCl₂) to make competent cells, electroporation, etc. Transfection of eukaryotic cells may be accomplished by a variety of means known to the art including, for example, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The terms “treat,” “treated,” or “treating” as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment. “Treatment” or “treating,” when referring to protection of a subject from a disease, may include suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof. In some embodiments, variants include homologues. Homologues may be polynucleotides or polypeptides or genes inherited in two species by a common ancestor.

As used herein, the term “vector” is used in reference to a polynucleotide that transfers polynucleotide segment(s) from one cell to another. A vector may also be referred to as a “vehicle” or a type of “polynucleotide construct” or “nucleic acid construct.” A vector may refer to a medium into which a polynucleotide sequence for encoding a desired protein can be inserted or introduced. A vector may refer to a polynucleotide molecule having nucleotide sequences that enable its replication in a host cell. A vector can also include nucleotide sequences to permit ligation of nucleotide sequences within the vector, wherein such nucleotide sequences are also replicated in a host cell. A vector can also mediate recombinant production of a polypeptide. Vectors include circular nucleic acid constructs such as plasmids, cosmids, viruses, etc., as well as linear nucleic acid constructs (e.g., lambda, phage constructs, PCR products), and other mediums. A vector may include expression signals such as a promoter and/or an enhancer, and in such a case it is referred to as an “expression vector.” The term “expression vector” as used herein refers to a polynucleotide molecule containing a desired coding sequence and appropriate polynucleotide sequences necessary for the expression of the operably linked coding sequence in a particular host organism. The expression vector can be transfected and into an organism to express a gene. The expression vector may be recombinant. A polynucleotide sequence for encoding a desired protein can be inserted or introduced into an expression vector. A vector may include polynucleotide sequences to promote or control expression in prokaryotes such as a promoter, an operator (optional), and a ribosome binding site, and other sequences. A vector may include polynucleotide sequences to promote or control expression in eukaryotes such as a promoter, enhancers, termination signal, and polyadenylation signal.

### 2. NEURONAL CELL ACTIVITY REPORTER SYSTEM

Further provided herein is a neuronal cell activity reporter system. The neuronal cell activity reporter system includes the SNAR construct, and a control construct.

a. Secreted Neuronal Activity Reporter (SNAR) Construct

Provided herein is a Secreted Neuronal Activity Reporter (SNAR) construct. The SNAR construct comprises a polynucleotide and includes an activity-dependent promoter and a polynucleotide encoding a first secreted reporter protein. In some embodiments, the SNAR construct comprises four tandem repeats of a core domain of the Synaptic Activity Response Element (SARE) of Arc/Arg3.1, the Arc minimal promoter, and a polynucleotide encoding a first secreted reporter protein. In some embodiments, the SNAR construct comprises a polynucleotide of SEQ ID NO: 12.

The SNAR construct may include a transcription element from an immediate early gene (IEG) in neurons. IEGs are a class of genes that are rapidly activated and can be transcribed in the presence of protein synthesis inhibitors. Upon stimulation, neurons rapidly induce the transcription of a number of IEGs. IEGs are induced by various neuronal stimuli, including electrical stimulations, environmental enrichment, sensory experience, and abusive drugs. Thus expression profiles of IEGs can be used to label the ensemble of activated neurons in a neuronal network. Domains of the activity-dependent enhancer and promoter element of several IEGs have been identified and may be engineered to generate activity-dependent drivers in constructs. For example, the robust activity marking system (RAM) is composed of four tandem repeats of a synthetic sequence derived from Npas4 and c-fos enhancers followed by the c-fos minimal promoter, and may be used to label active neuronal ensembles during memory encoding and recall (Sorensen, et al. eLIFE 2016, 5, e13918).

Examples of IEGs include c-fos, activity-regulated cytoskeleton-associated protein (Arc/Arg3.1), Homer1a, Egr-1, and Npas4. Arc/Arg3.1 is a plasticity protein and may have a role in learning and memory-related molecular processes. Arc/Arg3.1 is also a marker for intense synaptic activity. The synaptic activity response element (SARE) of Arc/Arg3.1 is an activity-dependent driver of transcription and enhancer element of the Arc/Arg3.1 gene. The SARE is approximately 100 bp in length and approximately 5-7 kb upstream of the Arc/Arg3.1 transcription initiation site. The SARE polynucleotide contains binding sites for cyclic AMP response element-binding protein (CREB), myocyte enhancer factor 2 (MEF2), and serum response factor (SRF). The SARE may promote rapid onset of transcription triggered by synaptic activity and low basal expression during synaptic inactivity. In some embodiments, the SARE of Arc/Arg3.1 comprises a polynucleotide of SEQ ID NO: 1. In some embodiments, the core domain of the SARE of Arc/Arg3.1 comprises a polynucleotide of SEQ ID NO: 2. In some embodiments, the SNAR construct comprises four tandem repeats of SEQ ID NO: 2.

The SNAR construct also includes a promoter. The promoter may be a polynucleotide comprising the Arc minimal promoter. In some embodiments, the Arc minimal promoter comprises a polynucleotide of SEQ ID NO: 3.

The SNAR construct also includes a polynucleotide encoding a first secreted reporter protein. The secreted reporter protein comprises a polypeptide that is secreted or exported from a cell and emits a detectable signal. In some embodiments, the secreted reporter protein emits a signal upon contact with at least one substrate. The substrate may be a luciferin. Luciferins are small molecules that emit light and may be found in organisms that generate bioluminescence. Substrates may include, for example, luciferin (such as Firefly luciferin), coelenterazine, furimazine (2-furanylmethyl-deoxy-coelenterazine), or a combination thereof. In some embodiments, the substrate is coelenterazine. In some embodiments, the first secreted reporter protein comprises Gaussia luciferase (which may be referred to as Gluc). The Gaussia luciferase may be rapidly secreted from the cell upon synthesis. The Gaussia luciferase may comprise a polypeptide comprising the amino acid sequence of SEQ ID NO: 7. Gaussia luciferase may comprise a polypeptide encoded by a polynucleotide of SEQ ID NO: 8. In some embodiments, the first secreted reporter protein comprises a polypeptide of SEQ ID NO: 7.

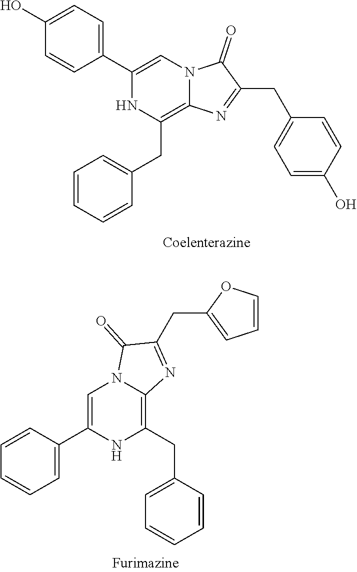 [SMILES: O=c1c(Cc2ccc(O)cc2)nc2c(Cc3ccccc3)[nH]c(-c3ccc(O)cc3)cn1-2 ; O=c1c(Cc2ccco2)nc2c(Cc3ccccc3)[nH]c(-c3ccccc3)cn1-2]

In some embodiments, the SNAR construct further includes sites suitable for recognition by or contact with a Cre recombinase. For example, the SNAR construct may include a loxP site, a lox2272 site, or a combination thereof. The loxP site may comprise a polynucleotide of SEQ ID NO: 4. The lox2272 site may comprise a polynucleotide of SEQ ID NO: 5. The SNAR construct may comprise a loxP site upstream of the polynucleotide encoding a first secreted reporter protein, and a lox2272 site downstream of the polynucleotide encoding a first secreted reporter protein. The SNAR construct may comprise a loxP site downstream of the polynucleotide encoding a first secreted reporter protein, and a lox2272 site upstream of the polynucleotide encoding a first secreted reporter protein.

A cell may be contacted with the SNAR construct. The SNAR construct may be administered to a cell. The cell may be from any type of subject. In some embodiments, the cell is human. In some embodiments, the cell is a mutant animal cell. The cell may be a neuronal cell, which may also be referred to as a neuron. The cell may be a neuron in or from the spinal cord. The cell may be a sensory neuron, motor neuron, or interneuron. The cell may be a neuron in or from the brain. The cell may be a cortex neuronal cell, a cerebellum neuronal cell, a retinal neuronal cell, and neuronal cells derived from other brain region such as striatum and midbrain. For example, the cell may be a neuronal cell derived from an embryonic pluripotent stem cell or an induced pluripotent stem cell (iPSC). The cell may be a live cell. Contacting or administering may include any suitable method known in the art such as, for example, infection, transfection, transduction, transformation, and electroporation.

The SNAR construct may be any or introduced into any suitable type of vector known in the art. For example, the SNAR construct may be plasmid, a vector, a viral vector, an adeno-associated virus (AAV), or a lentivirus. The SNAR construct may be recombinant.

b. Control Construct

The control construct comprises a polynucleotide and includes a constitutive promoter and a polynucleotide encoding a second secreted reporter protein. The constitutive promoter may comprise a human PGK promoter or any other promoter that is not affected by neuronal activity. The human PGK promoter may comprise a polynucleotide of SEQ ID NO: 6. In some embodiments, the control construct comprises a polynucleotide of SEQ ID NO: 13.

In some embodiments, the second secreted reporter protein emits a signal upon contact with a substrate, the signal being distinct from the signal emitted by the first secreted reporter protein upon contact with a substrate, as detailed above. In some embodiments, a first substrate such as furimazine (FMZ) reacts specifically with the second secreted reporter protein but does not cross-react with the first secreted reporter protein. In some embodiments, a second substrate such as coelenterazine (CTZ) reacts with both the first secreted reporter protein and the second secreted reporter protein.

In some embodiments, the second secreted reporter protein comprises a nanoluciferase. In some embodiments, the second secreted reporter protein comprises a nanoluciferase having an N-terminal secretion signal peptide, which may be referred to as secreted nanoluciferase (sNluc). A secretion signal peptide guides or signals the polypeptide to which it is attached to be exported from a cell. In some embodiments, the N-terminal secretion signal peptide comprises a polypeptide of SEQ ID NO: 11 (METDTLLLVVVLLLVVVPGSTGD). The secreted nanoluciferase may comprise a polypeptide comprising the amino acid sequence of SEQ ID NO: 9. Secreted nanoluciferase may comprise a polypeptide encoded by a polynucleotide of SEQ ID NO: 10. In some embodiments, the second secreted reporter protein comprises a polypeptide of SEQ ID NO: 9.

In some embodiments, the control construct further includes sites suitable for recognition by or contact with a Cre recombinase. For example, the control construct may include a loxP site, a lox2272 site, or a combination thereof. The loxP site may comprise a polynucleotide of SEQ ID NO: 4. The lox2272 site may comprise a polynucleotide of SEQ ID NO: 5. The control construct may comprise a loxP site upstream of the polynucleotide encoding a second secreted reporter protein, and a lox2272 site downstream of the polynucleotide encoding a second secreted reporter protein. The control construct may comprise a loxP site downstream of the polynucleotide encoding a second secreted reporter protein, and a lox2272 site upstream of the polynucleotide encoding a second secreted reporter protein.

The control construct may be any or introduced into any suitable type of vector known in the art. For example, the control construct may be plasmid, a vector, a viral vector, an adeno-associated virus (AAV), or a lentivirus. The control construct may be recombinant.

### 3. METHODS OF MONITORING NEURONAL ACTIVITY

Provided herein is a method of monitoring neuronal activity in a test cell. In some embodiments, the method includes administering to the test cell the SNAR construct as detailed herein. The first secreted reporter protein, or the test cell comprising the first secreted reporter protein, may be contacted with a substrate, wherein the substrate reacts with the first secreted reporter protein to generate a first signal (CTZₛₐₘₚₗₑ). The substrate may be coelenterazine. The first signal is measured.

The neuronal activity in the test cell may be determined based on the first signal. In some embodiments, the first signal is measured at two different time points, and the neuronal activity in the test cell at the two different time points are compared. In some embodiments, the neuronal activity in the test cell is monitored by measuring the first signal at a plurality of different time points. The first secreted reporter protein may be exported out of the test cell to a culture medium. The first secreted reporter protein may be contacted with the substrate by adding the substrate to a sample of the culture medium.

In other embodiments, a control construct as detailed herein may be incorporated. The method may include administering to the test cell the neuronal cell activity reporter system as detailed herein. As detailed above, the neuronal cell activity reporter system includes a SNAR construct and a control construct. The first secreted reporter protein and the second secreted reporter protein in the test cell, or the test cell comprising the first and second secreted reporter proteins, may be contacted with a first substrate, wherein the first substrate reacts with the first secreted reporter protein and the second secreted reporter protein to generate a first signal (CTZsample). The first substrate may be coelenterazine. The first signal is measured. The first secreted reporter protein and the second secreted reporter protein in the test cell, or the test cell comprising the first and second secreted reporter proteins, may be contacted with a second substrate, wherein the second substrate reacts with the second secreted reporter protein to generate a second signal (FMZₛₐₘₚₗₑ). The second substrate may be furimazine. The second signal is measured.

In these embodiments, the method may further include determining a control ratio. To determine a control ratio, the method further includes administering to a control cell a control construct as detailed herein. The second secreted reporter protein from the control cell may be contacted with the first substrate, wherein the first substrate reacts with the second secreted reporter protein to generate a third signal (CTZ_{sNluc}). The third signal is measured. The second secreted reporter protein from the control cell may be contacted with the second substrate, wherein the second substrate reacts with the second secreted reporter protein to generate a fourth signal (FMZ_{sNluc}). The fourth signal is measured. As indicated above for the test cell, the first substrate may be coelenterazine, and the second substrate may be furimazine. For example:
Gluc=first secreted reporter protein;
sNluc=second secreted reporter protein;
CTZₛₐₘₚₗₑ=first signal, from the first and second secreted proteins;
FMZₛₐₘₚₗₑ=second signal, exclusively from the second secreted protein;
CTZ_{sNluc}=third signal, from a sample with control construct only;
FMZ_{sNluc}=fourth signal, from a sample with control construct only.

The control ratio is determined by dividing the third signal by the fourth signal (CTZ_{sNluc}/FMZ_{sNluc}). The neuronal activity in the test cell may be determined based on the contribution of the first secreted reporter protein to the first signal with the control ratio factored in. In such embodiments, the contribution of the first secreted reporter protein to the first signal is calculated by subtracting from the first signal the product of the control ratio and the second signal (first signal−(third signal/fourth signal]×second signal]=CTZₛₐₘₚₗₑ−[(CTZ_{sNluc}/FMZ_{sNluc})×FMZₛₐₘₚₗₑ]) (Heise, et al. Assay Drug Dev. Technol. 2013, 11, 244-252). In an assay, there may be multiple test cells or samples, and at least one control cell or sample.

In embodiments wherein the second secreted reported protein contributes less than 10%, less than 9%, less than 8%, less than 7%, less than 6%, less than 5%, less than 4%, less than 3%, less than 2%, or less than 1% to the first signal, a control ratio may not be needed or necessary to accurately determine the contribution of the first secreted reporter protein to the first signal. For example, in embodiments wherein the changes in the first signal are monitored within the same sample over time, the introduction of the second secreted protein may not be needed or necessary to accurately determine the changes in the neuronal activity.

In some embodiments, the contribution of the second secreted reported protein to the first signal may be great enough that a control ratio may be used to control for and more accurately determine the contribution of the first secreted reporter protein to the second signal. For example, the second secreted reported protein (for example, sNluc) may contribute more than 2%, more than 3%, more than 4%, more than 5%, more than 6%, more than 7%, more than 8%, more than 9%, or more than 10% to the first signal, such that a control ratio may be used to control for and more accurately determine the contribution of the first secreted reporter protein to the first signal. In such embodiments, the method may further include determining a control ratio, as detailed above, and administering to the test cell a control construct (in addition to a SNAR construct) and administering to a control cell a control construct (and no SNAR construct) as detailed herein. For example, in embodiments wherein the absolute amount of the reporter proteins (not the change in reporter proteins over time) is compared between samples or wells, both the first and second reporter proteins are introduced to cells. In embodiments wherein neuronal activities are compared between samples or wells in a fresh medium after complete washout of a pre-conditioned medium, the ratio of the first reporter protein to the second reporter protein is compared. When neurons are cultured in different conditions from the beginning or bear mutation, the ratio of the first reporter protein to the second reporter protein is compared between samples or wells.

The cells may be cultured in a culture medium. The cells may be maintained in any suitable culture medium, temperature, oxygen conditions, humidity conditions, and/or pressure in order to keep the cells alive. For example, the cells may be maintained in a humidified incubator.

The first secreted reporter protein and the second secreted reporter protein may be exported out of the test cell to a culture medium. The secreted reporter proteins may accumulate in the culture medium over time. A sample of the culture media may be mixed with the substrate to generate the first signal, the second signal, the third signal, the fourth signal, or a combination thereof. In some embodiments, the first secreted reporter protein and the second secreted reporter protein are contacted with furimazine by adding furimazine to a sample of the culture medium. In some embodiments, the first secreted reporter protein and the second secreted reporter protein are contacted with coelenterazine by adding coelenterazine to a sample of the culture medium.

In some embodiments, the method further comprises contacting the test cell with a modulator of synaptic signaling. The modulator may be an inhibitor or an effector of neurons. The modulator may be an antagonist or an agonist of neurons, such as an antagonist or an agonist of neuron growth, function, activity, signaling, differentiation, or a combination thereof. Modulators of synaptic signaling may include, for example, factors from astrocyte conditioned media (ACM), TTX, AP5, CNQX, dopamine, serotonin, acetylcholine, histamine, norepinephrine, drugs such as epilepsy drugs, polypeptides, proteins, small molecules, agonists of synaptic receptors, antagonists of synaptic receptors, and derivatives thereof.

The first signal and the second signal may be measured at two different time points. The neuronal activity in the test cell at the two different time points may be compared. The neuronal activity in the test cell may be monitored by measuring the first signal and the second signal at a plurality of different time points. The plurality of time points may be every 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 120 minutes, 3 hours, 4 hours, 5 hours, 6 hours, 12 hours, 24 hours, 36 hours, 48 hours, 3 days, 4 days, 5 days, 6 days, 1 week, 1.5 weeks, or 2 weeks. The plurality of time points may be taken over the course of 2 seconds, 3 seconds, 4 second, 5 seconds, 10 seconds, 20 seconds, 30 seconds, 40 seconds, 45 seconds, 50 seconds, 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 120 minutes, 3 hours, 4 hours, 5 hours, 6 hours, 12 hours, 24 hours, 36 hours, 48 hours, 3 days, 4 days, 5 days, 6 days, 1 week, 1.5 weeks, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 1 month, 2 months, 3 months, 4 months, 5 months, 6 months, 7 months, or 8 months.

As indicated above the SNAR construct and/or the control construct may include sites suitable for recognition by or contact with a Cre recombinase such as a loxP site and a lox2272 site. In such embodiments, expression of the secreted reported protein may be selective and conditional. For example, the secreted protein may only be expressed in cells with the construct upon administration of the Cre recombinase. The neuronal activity of only a subpopulation of neurons may be monitored, while maintaining the activity of other types of neurons.

As indicated above, the test cell may be a live cell, and the control cell may be a live cell. The signals may be measured without harvesting or killing the cell.

### 4. EXAMPLES

### Example 1

### Materials and Methods

Animals. All animal care and experiments were conducted in accordance with NIH guidelines and approved by IACUC (University of Utah protocol #15-03003). C57Bl6/J mouse lines were maintained under the normal housing conditions with food and water available ad libitum and 12:12 h light-dark cycle at the University of Utah.

Statistical Analyses. All data were analyzed by two-tailed Student's t test unless otherwise stated. A one-way ANOVA was used followed by Bonferroni multiple comparisons where more than two conditions were tested (Bonferroni p-values shown). All data are shown as mean±S.E.M. No statistical methods were used to pre-determine sample sizes but our sample sizes were similar to those generally employed in the field. Formal randomization and blinding were not performed, although cell cultures were chosen randomly for each experimental group and data were objectively collected and analyzed. For all experiments, the n numbers shown refer to the number of wells used per condition over at least three separate cultures, otherwise mentioned specifically.

Cell Culture. Astrocyte cultures were prepared from wild-type (WT) mice aged P2 using the traditional method. Briefly, cortices were dissected, enzymatically (using 165 units of papain, Worthington LS003126) and mechanically digested until a single cell suspension is obtained. Cells were plated in poly-D-lysine (Millipore, Burlington, Mass.; catalog no. A-003-E) coated flasks and grown in glial media containing MEM (Mediatech 15-010-CV), 10% horse serum and 1% Penicillin/Streptomycin/Glutamine (Invitrogen, Waltham, Mass.; catalog no. 10378-016), until confluent. Other cell types were removed by shaking.

Neuronal cultures were prepared from PO WT mice using a similar protocol. Neuronal cultures were prepared from hippocampi or forebrain and cultured in poly-L-lysine (Sigma Aldrich, St. Louis, Mo.; catalog no. P2636) coated plates or coverslips. Cultures were treated once with AraC (2.5 μM, Sigma Aldrich, St. Louis, Mo.; catalog no. C6645) after 4 days in vitro (DIV) to prevent proliferation of astrocytes. In experiments testing the effect of astrocyte-conditioned media, AraC treatment was performed on DIV1. Half of the media was replaced every 3 days. Neuronal media (Neurobasal A containing 1% horse serum, 1% Glutamax, 2% B-27 and 1% Penicillin/streptomycin) for this purpose was incubated in confluent astrocyte cultures overnight. In experiments testing the effect of astrocyte-conditioned media, 50 μg of total protein was added per well. Neuronal cultures were collected at DIV14-164 for immunocytochemistry.

HEK293T cells were cultured in DMEM containing 10% FBS (Invitrogen, Waltham, Mass.; catalog no. 16140071), 1% sodium pyruvate (Invitrogen, Waltham, Mass.; catalog no. 11360070) and 1% Penicillin/Streptomycin (Invitrogen, Waltham, Mass.; catalog no. 15140122). Cells were plated at a density of 0.2 million cells per well onto a PEI (25 μg/μL) coated 12-well plate and transfected the next day using the Fugene 6 transfection reagent and according to the manufacturer's directions. After 24 hours, media was completely changed to serum free DMEM and approximately 24 hours later both media and lysates were harvested. Media samples were stored short term at 4° C. until used for luciferase assays.

Lentivirus Packaging. Lenti-X 293T cells were cultured in DMEM containing 10% FBS, 1% sodium pyruvate and 1% Penicillin/Streptomycin. At approximately 90% confluence, cells were plated at a density of 2.5-3 million cells per 10 cm dish and transfected the next day using Fugene 6. Packaging plasmids pMD2.G and psPAX2 were obtained from Addgene (Watertown, Mass.; plasmids #12259 and 12260 respectively) and used at this ratio (10 μg:6 μg:10 μg, transfer:pMD2.G:psPAX2). 24 hours after transfection the media was completely replaced and plates returned to the incubator for an additional 48 hours. The media was collected and filtered through a 0.45 μm PES filter. Lentiviral supernatant was then centrifuged using a benchtop Beckman Optima XP ultracentrifuge at 120,000 g for 2 hours at 4° C. The lentiviral pellet was then resuspended in DPBS and aliquots stored at −80° C. until needed.

Immunocytochemistry and Imaging. Cells were rinsed with DPBS and immediately fixed using 4% paraformaldehyde with 4% sucrose in PBS for 15 minutes. After rinsing with PBS and permeabilization using 0.2% Triton-X100 in PBS, a 1 hour block with 4% BSA and 4% normal goat serum in PBS, the cells were incubated overnight at 4° C. in primary antibody. Primary antibodies used are listed in TABLE 1. After washing with PBS, the cells were incubated in secondary antibody. Coverslips were mounted using Prolong gold mounting solution. Imaging was performed using either a Nikon E800 epi-fluorescence microscope or Nikon A1 for confocal imaging.

TABLE 1
Primary antibodies.

| | | Company | |
|---|---|---|---|
| Antigen | Species | Cat. No. | Dilution |
| CamKII | mouse | Millipore 05-532 | 1:5,000 |
| Gluc | rabbit | Nanolight 401P | 1:1,000 |
| GAD67 | mouse | Millipore MAB5406 | 1:1,000 |
| GFAP | mouse | Cell Signaling 3670 | 1:500 |
| MAP2 | chicken | Abcam ab5392 | 1:10,000 |

Plasmid Cloning. The SNAR construct was synthesized by first introducing 2 core SARE (cSARE) sequences into an AAV vector backbone that included the Arc minimal promoter and the Gaussia luciferase coding sequences using inFusion cloning. A single cSARE sequence was synthesized using a long primer that was then used as a PCR template. Two additional cSARE sequences were then added, one at a time preceding the first 2 cSAREs. The entire 4× cSARE-ArcMin-Gluc was then cloned as an insert into an FCK vector (Addgene, Watertown, Mass.; catalog no. 51694) using restriction enzymes PacI and EcoRI for use as a transfer plasmid and packaging into lentivirus particles. Secreted Nanoluciferase (sNluc) was obtained by adding the Ig-kappa signal peptide to its N-terminus using a long primer. sNluc was then inserted into an FCK vector with the hPGK promoter. Each construct was then cloned into a DIO vector backbone (Addgene, Watertown, Mass.; catalog no. 87168) to obtain a Cre-dependent expression construct. Floxed constructs were then inserted back into the same FCK vector for lentivirus packaging.

Luciferase Assays. To determine luciferase activity luminescencence from media samples was measured. For samples, 10-20 μL of conditioned media were loaded onto the wells of a 96-well opaque white plate. For substrates, Coelenterazine (CTZ)-native (NanoLight Technology, Pinetope, Ariz.; catalog no. 303) and FMZ (Promega NanoGlo Assay; Promega, Madison, Wis.; catalog no. N1110) were added using a micro-injector connected to the plate reader (BioTek Synergy HT; Winooski, Vt.). CTZ was dissolved in acidic ethanol before the use.

Reaction was initiated by adding substrate, CTZ (NanoLight Technology, Pinetope, Ariz.) into cell lysates or medium as indicated, and luciferase signal was measured by a microplate reader (BioTek, Winooski, Vt.).

### Example 2

### Secreted Neuronal Activity Reporter (SNAR) and Dual Secreted Luciferase Assay

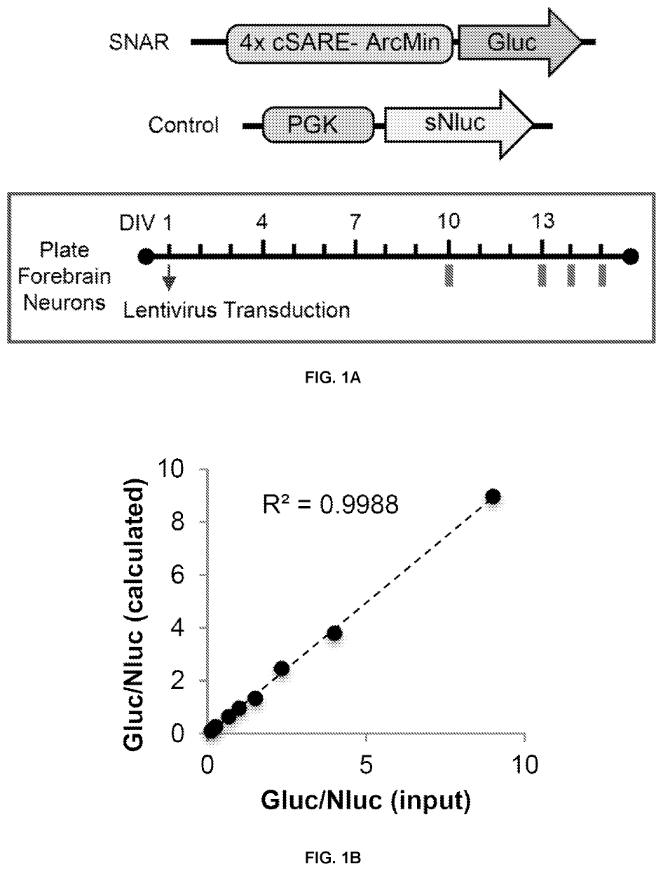

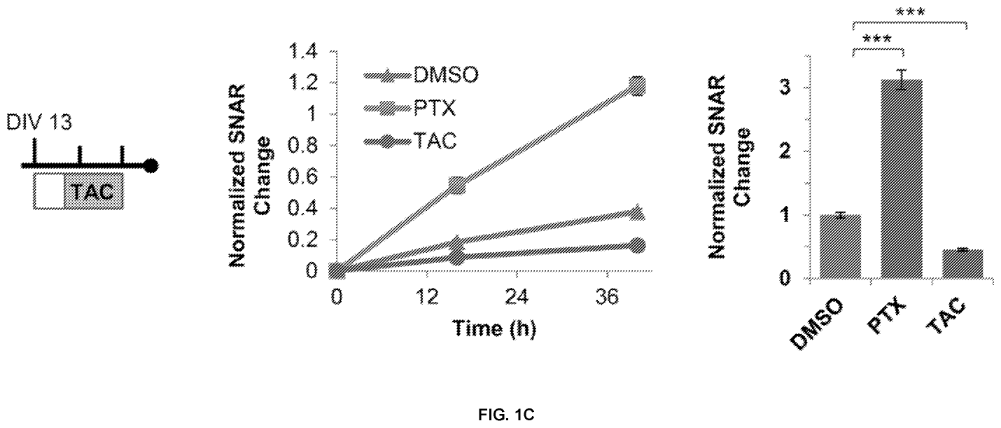

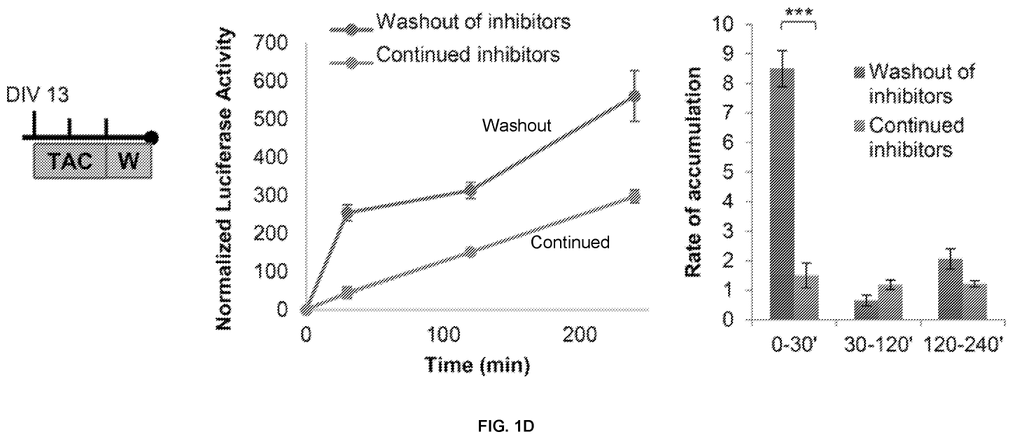

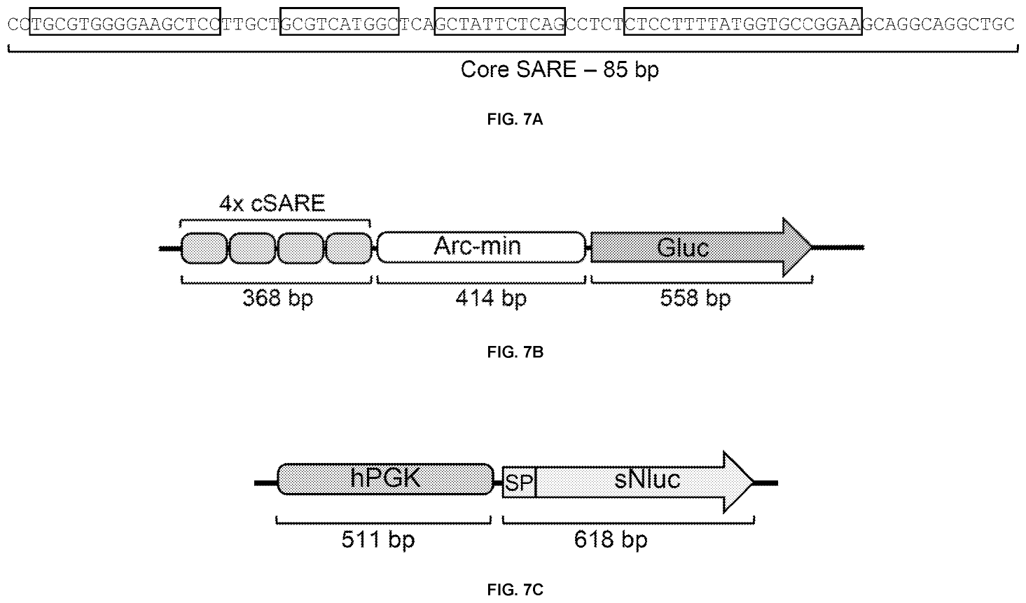

The enhancer element of the immediate early gene Arc/Arg3.1, namely the synaptic activity response element (SARE), has been exploited as an activity-dependent driver (Kawashima, et al. Nat. Methods 2013, 10, 889-895) (Das, et al. Sci. Adv. 2018, 4, eaar3448) (Wu, et al. Neurosci. Lett. 2018, 666, 92-97). We took the conserved core element from the previously characterized SARE sequence in order to make a compact reporter efficiently delivered using a variety of approaches including both AAV and lentivirus (FIG. 7A-FIG. 7C) (Kawashima, et al. Proc. Natl. Acad. Sci. USA 2009, 106, 316-321). We combined four tandem repeats of the core domain of SARE and the Arc minimal promoter (hereafter called an activity-dependent driver). We combined the activity-dependent driver with Gaussia luciferase (Gluc), which is rapidly secreted from the producing cells upon synthesis, and named the reporter cassette as Secreted Neuronal Activity Reporter (SNAR) (FIG. 1A and FIG. 7B). As a control we converted Nanoluciferase into a secreted protein by inserting a signal peptide preceding its N-terminus. Secreted Nanoluciferease (sNluc) was then coupled with the human PGK promoter to make a secreted control (FIG. 1A and FIG. 7C). Both Gluc and Nluc are small and the brightest luciferases available.

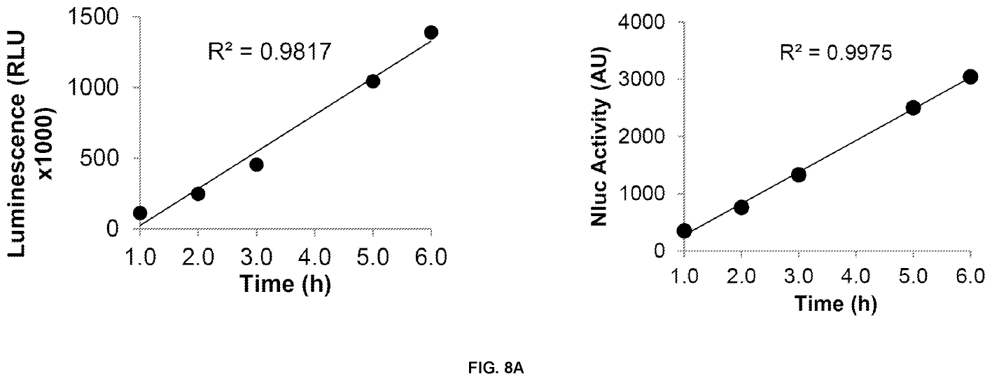

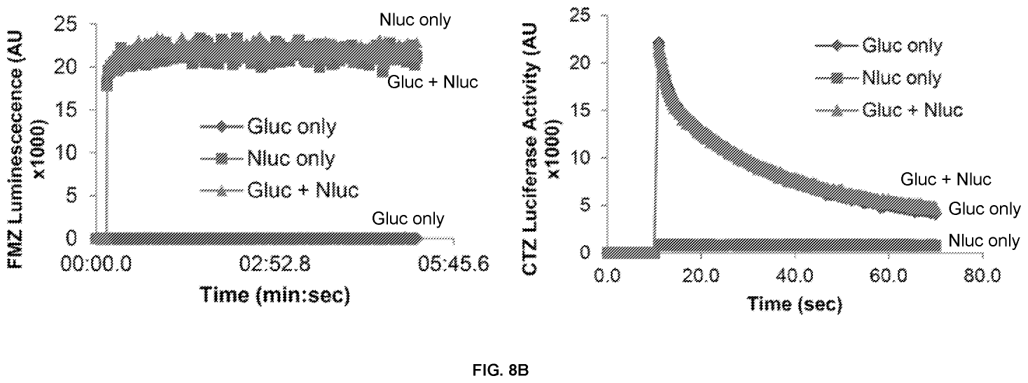

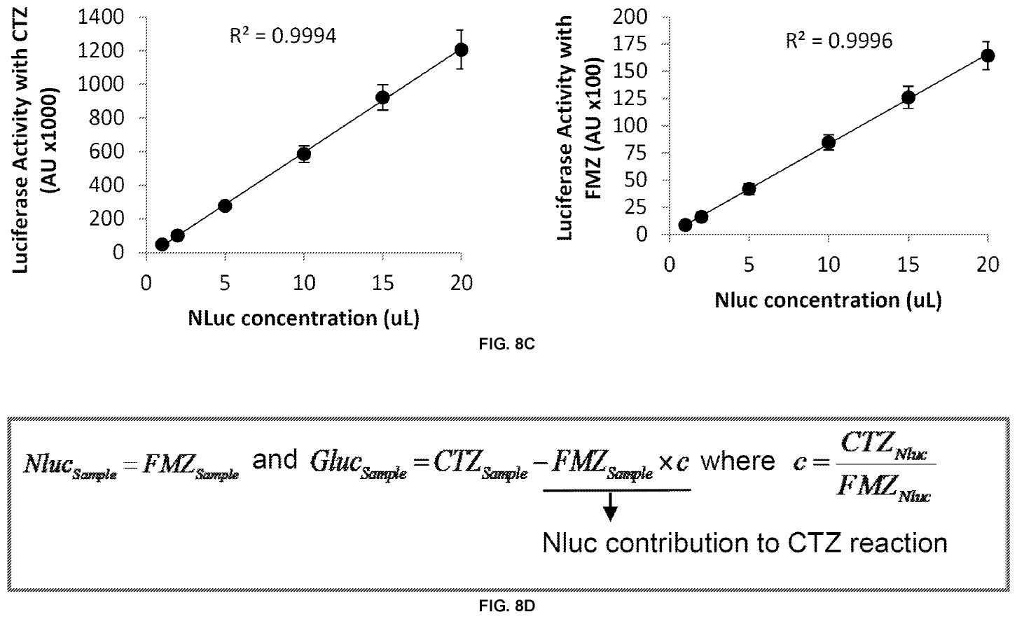

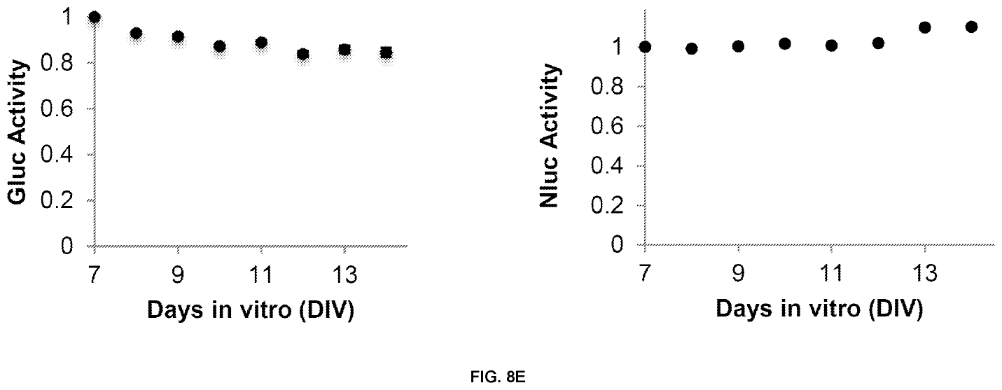

Although both Gluc and Nluc are a Renilla-type luciferase, they have distinct kinetics and substrate specificity and can be combined as a dual luciferase system. To determine whether we could independently measure Gluc and sNluc in our luciferase assay, we first tested this using the culture medium of 293T cells transfected with Gluc or sNluc under a constitutive promoter (pCAG). Furimazine (FMZ) substrate reacted specifically with sNluc and does not cross-react with Gluc (FIG. 8B). Coelenterazine (CTZ), which is a robust substrate for Gluc, also reacted with sNluc albeit at low level (FIG. 8B). The bioluminescence of sNluc in CTZ reaction (CTZ_{sNluc}) was linearly proportional to the amount of sNluc in the sample (FIG. 8A-FIG. 8E). Thus we were able to separate the contribution of Gluc (CTZ_{Gluc}) and Nluc (CTZ_{Nluc}) in the CTZ reaction of a mixed sample (CTZₛₐₘₚₗₑ). We first obtained the ratio (c) of the bioluminescence of sNluc only samples in CTZ reaction to FMZ reaction (c=CTZ_{sNluc}/FMZ_{sNluc}). After measuring the bioluminescence of a mixed sample in both CTZ (CTZ_{Sample}) and FMZ (FMZ_{Sample}) reactions, we calculated the contribution of sNLuc to the CTZ signal by multiplying FMZ_{Sample} by the constant ratio (c). By simply subtracting the contribution of sNluc from the total CTZ signal, we calculated the contribution of Gluc to the CTZ reaction (Gluc_{Sample}=CTZ_{Sample}−c×FMZ_{Sample}) (FIG. 8D). Using this method, we reliably determined the Gluc/Nluc ratio in a mixed sample that reflects the input Gluc/Nluc ratio (FIG. 1B, slope=0.9944, R²=0.9988).

The kinetic properties of each luciferase were not significantly affected by the other, as shown in kinetic plots of samples containing both luciferases (FIG. 8B) further validating the quantitative measurement of each luciferase in a dual luciferase assay. Both luciferases linearly accumulated in the medium over time in naïve condition (FIG. 8A), suggesting that the slope of the reporter accumulation in the medium reflects the synthesis rate of the reporter inside the producing cells. To determine the stability of the secreted Gluc and sNluc in the culture medium, conditioned media was transferred from transfected cells to a non-transfected well, and the decaying kinetics of bioluminescence was measured over days (FIG. 8E). Both Gluc and sNluc were stable over several days.

### Example 3

### SNAR Reflects Neuronal Activity

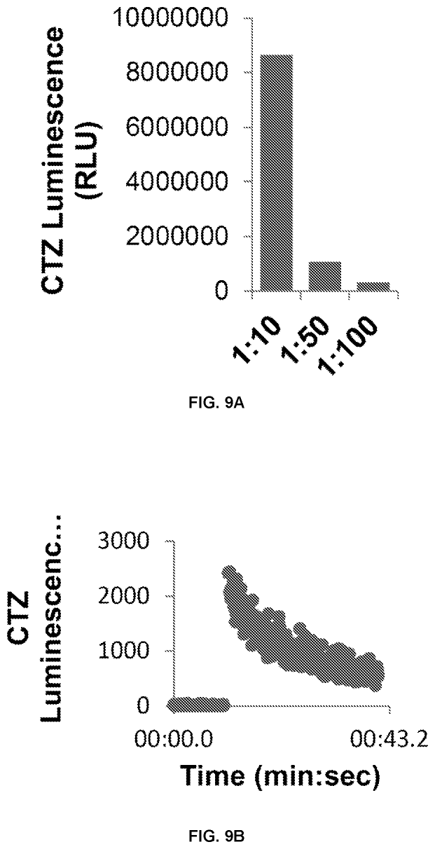

To determine whether the SNAR could be used to reliably monitor neuronal activity, it was first tested if manipulating neuronal activity would lead to changes in the reporter activity. Primary forebrain neuronal cultures were infected with lenti-SNAR and lenti-pPGK:sNluc after one day in vitro (DIV1) and were cultured in normal culture conditions. To inhibit neuronal activity, neurons were treated with a cocktail of inhibitors (2 μM TTX, 200 μM AP5, and 8 μM CNQX: TAC) on DIV13, and the reporter activity was measured over time. It was observed that the assay can be performed with a very small volume of media, a fraction of 1 μL, thus allowing for multiple time points to be collected without significant changes in the culture conditions (FIG. 9A-FIG. 9B). Reporter accumulation in the medium was similar at 16 hours after the inhibitors were added. After 16 hours, there was a significant reduction in the slope of SNAR accumulation in the presence of the inhibitors (FIG. 1C). The delayed response to the inhibitors was likely due to ongoing release of pre-synthesized protein in the secretory pathway and continued protein synthesis from pre-existing transcript. To reveal the effect of the inhibitors after the lagging time, the inhibitors were pretreated for 16 hours and the fold increase in SNAR activity in the culture medium during the following 24 hours was quantified. Blocking neuronal activity in primary neurons using a cocktail of inhibitors (TAC), dramatically decreased reporter activity (78.14% decrease in SNAR activity, FIG. 1C). Conversely, stimulation of neurons by washout of the inhibitors rapidly induced SNAR activity as normalized by pPGK:sNluc within 30 minutes of stimulation (5.7+/−1.22 fold increase as normalized by PGK:sNluc, p<0.001)(FIG. 1D). Notably, temporal analysis of SNAR revealed that 30 minutes after stimulation, the reporter activity was reduced to the basal rate, demonstrating the spike-like promoter activity of an immediate early gene upon neuronal stimulation. Overall, dual secreted reporter system of SNAR and pPGK:sNluc reliability monitored changes in the neuronal activity in live neurons.

### Example 4

### Longitudinal Measurement of Neuronal Activity

Next, it was tested if the reporter could be used to monitor development of neuronal activity over longer time periods. This would be a substantial advantage over existing techniques since it would allow the study of the kinetics of pharmacological agents over time. In addition, this would allow use of the reporter activity as a proxy for synapse formation and potentially identify modulators of this process with temporal specificity.

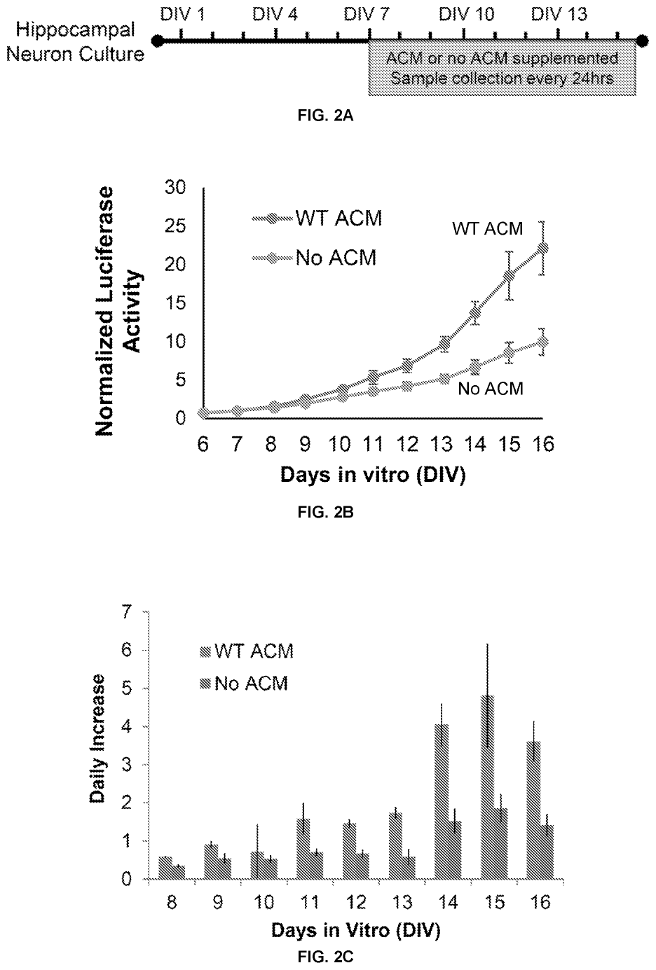

Astrocyte conditioned media (ACM) contains diffusible factors, both known and unknown, that promote synapse formation and modulate synaptic activity. Hence, it was tested if SNAR could reveal the role of astrocyte-derived factors in synapse development. Wild-type neurons were first treated with either ACM or unconditioned medium (no ACM), and reporter activity was monitored daily until neurons reached maturity (DIV16) (Chanda, et al. J. Neurosci. 2017, 37, 6816-6836). In both conditions a gradual increase in reporter activity was observed, consistent with neuronal maturation and increased synapse number. The two conditions became significantly different from DIV11 (p=0.019) and the difference increased over the following days (FIG. 2A). The largest increase occurred between DIV 14-15 (FIG. 2B and FIG. 2C), showing that astrocyte-derived factors facilitated the neuronal activity at both early and maturation stages, which lead to bigger functional consequences at the later stage.

### Example 5

### Temporal Analysis of Pharmacological Manipulations

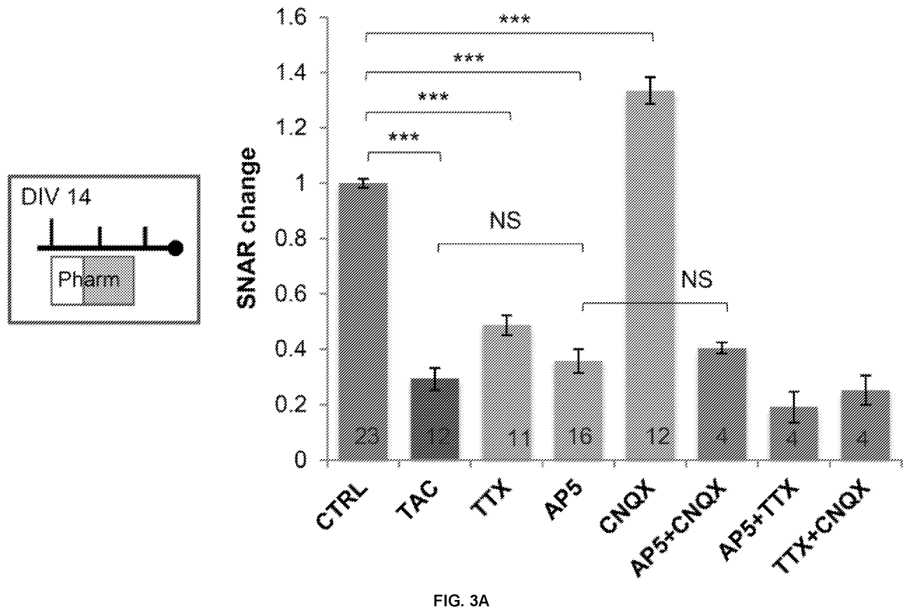

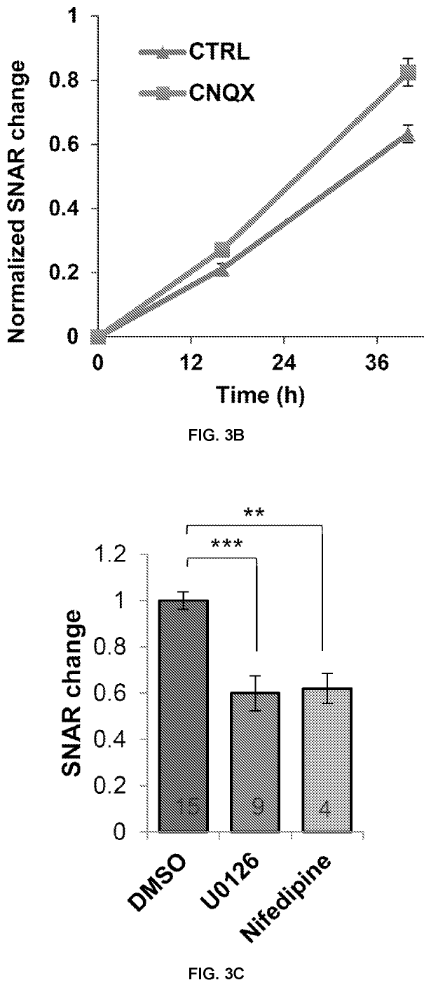

Inhibition of the neuronal firing and synaptic inputs by an inhibitor cocktail, which includes TTX, AP5, and CNQX, suppressed SNAR activity (FIG. 1B). To determine the role of specific synaptic inputs in SNAR activity, the effect of individual inhibitors on SNAR activity was tested. Inhibition of NMDAR-mediated transmission by AP5 treatment dramatically suppressed SNAR activity (FIG. 3A, Bonferroni pval<0.001). The reduction of SNAR activity by TTX alone was smaller than AP5 alone, indicating that the residual activity that was not suppressed by TTX was likely mediated by NMDAR signaling stimulated by spontaneous glutamate release or tonic NMDAR transmission by ambient glutamate. Interestingly, blockage of AMPAR transmission by CNQX treatment increased SNAR activity (Bonferroni pval<0.001). Although previous studies reported that CNQX paradoxically increases the expression of endogenous Arc (Rao, et al. Nat. Neurosci. 2006, 9, 887-895), the underlying mechanism was not known. Since the assay allow for multi-time point analysis, the time course of CNQX effect on SNAR activity was characterized. Although both prolonged CNQX treatment (FIG. 3A) and the stimulation of synaptic inputs (FIG. 1D) induced SNAR activity, unlike the synaptic stimulation, which occurred within 30 minutes (FIG. 1D), CNQX treatment did not induce SNAR activity until 16 hours of stimulation. The increase in the SNAR activity was observed between 16-40 hours after the treatment, showing the delayed response of neuronal activity to the chronic blockage of AMPAR-mediated transmission (FIG. 3B). Prolonged inactivity may have led to homeostatic adaptation, which may be accompanied by an increase in the expression and surface delivery of GluN1, GluN2A and GluN2B, major subunits of NMDAR, synaptic delivery of GluN2A-containing NMDAR, and NMDAR transmission. Notably, the magnitude of tonic NMDAR current mediated by ambient glutamate was dramatically enhanced by prolonged network inactivity. An increase in the SNAR activity during chronic blockage of AMPAR may be mediated by an increase in NMDAR transmission. Indeed, CNQX+AP5 co-treatment completely blocked the CNQX effect to the same level of AP5 alone (FIG. 3A). Overall, this assay not only detected the acute effect but also revealed the homeostatic response of neurons to prolonged drug treatment.

To test the intracellular signaling pathways on SNAR activity, we treated neurons with the ERK1/2 inhibitor, U0126 (10 μM) (Kawashima et al. Proc. Natl. Acad. Sci. USA 2009, 106, 316-321). U0126 significantly suppressed SNAR accumulation suggesting MAPK signaling also contributes to reporter activity (FIG. 3C). These results were consistent with previous studies showing both Arc and MAPK signaling play roles in synaptic plasticity.

### Example 6

### SNAR as a Screening Tool

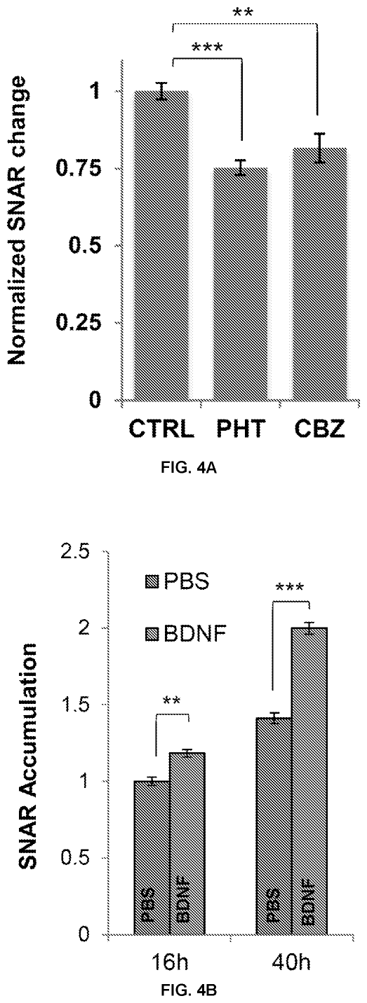

It was observed that the reporter was consistent and able to detect even small changes (about 10% changes from the control) with statistical significance. To test the potential usage of SNAR assay as screening tool, the consistency, robustness, and the separation band width of the assay were characterized. To determine whether the assay would be useful to identify modulators for synaptic signaling, the condition of an inhibitor cocktail (TAC) was used as a maximum range of synaptic inhibition (background condition). The assays showed 10.5 of signal to noise ratio (S/N), 4.73 of signal to background ratio (S/B), and 0.32 of Z-score, which is in a useful screen category. Next it was tested if drugs that have been characterized to modulate neuronal activity would have been identified by SNAR screens. It was found that neurons treated with 80 μM phenytoin, an anti-epilepsy drug commercialized as Dilantin and thought to act as a sodium channel blocker, had significantly reduced reporter activity at DIV14 (PHT p<0.001, CBZ p<0.01, FIG. 4A). Importantly, all four repetitions of PHT treatment fell outside the two standard deviation cutoff of the untreated condition, which provided 99% of confidence of hits without the replication of the drug screen, showing the usefulness of the SNAR assay as a drug screen tool. Conversely, treatment of neurons with BDNF, a neurotropic factor that enhances synapse formation and transmission, significantly and robustly increased reporter activity compared to a vehicle control (FIG. 4B). Notably, unlike the delayed enhancement of the reporter activity by chronic blockage of AMPAR-mediated transmission (FIG. 3B), the effect of BDNF was detected even at 16 h after start of treatment and was further enhanced 40 h later. These results suggested that the mechanism by which a neurotropic factor enhances neuronal activity is distinct from that by chronic blockage of the network activity. SNAR assay was able to distinguish the acute versus delayed effects of pharmacological manipulation and provided mechanistic insights even from the initial screens.

### Example 7

### Cell-Type Specific Expression

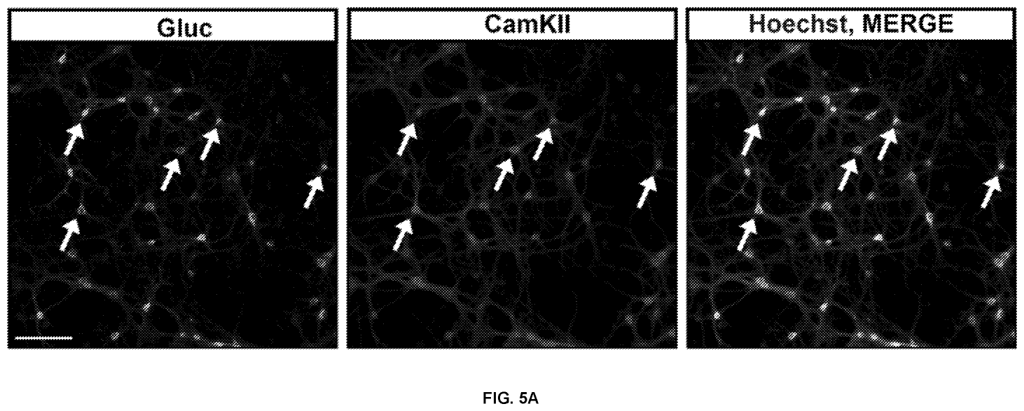

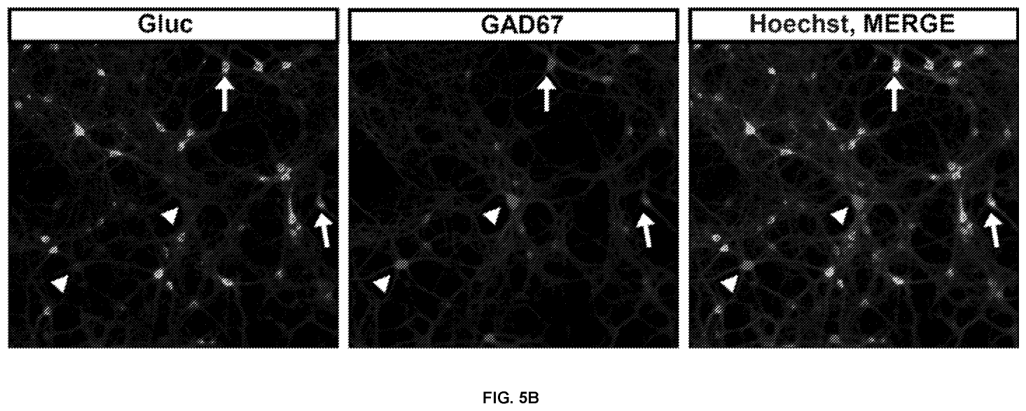

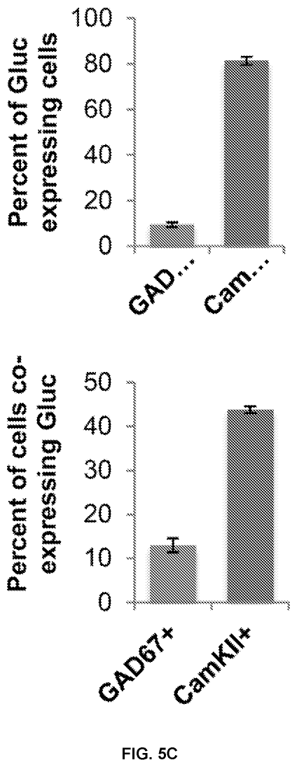

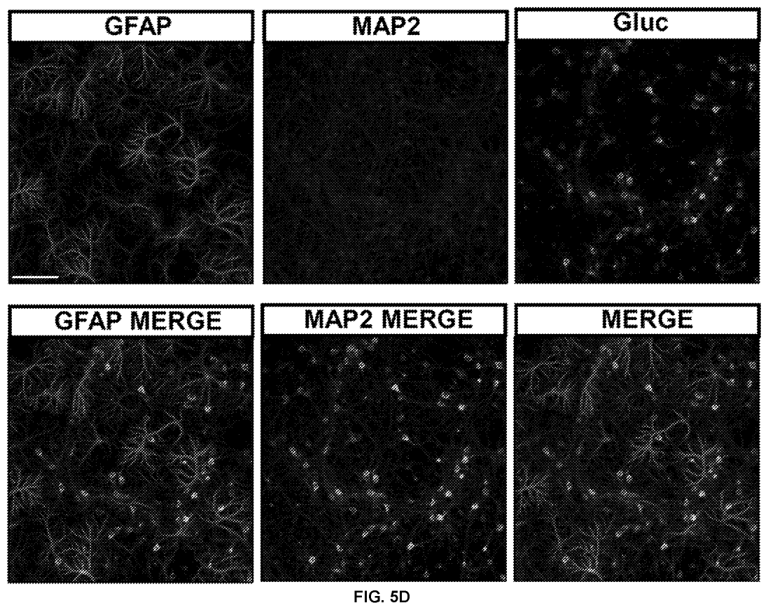

Primary neuronal cultures are comprised of different cell types including excitatory and inhibitory neurons. To determine the identity of cells expressing the SNAR reporter, we performed immunostaining of Gluc together with cell type specific markers. Consistent with the expression of endogenous Arc, the majority (˜80%) of cells expressing SNAR (Gluc-positive) were Cam KII-expressing excitatory neurons (FIG. 5A, 5B). Surprisingly, we also found that a small subpopulation of inhibitory neurons (9.37% of GAD67-positive cells) also expressed the reporter (FIG. 5B arrows). To confirm that this was not just due to the relative abundance of excitatory neurons in culture, as compared to inhibitory neurons, we also quantified the portion GAD67+ neurons that expressed the reporter. We found that a similar percentage of GAD67+ neurons expressed SNAR (FIG. 5B). To ensure that some of the Gluc-positive and Cam KII-negative cells were not astrocytes, we also performed immunostaining for a common astrocyte marker, GFAP. We prepared a mixed culture where astrocytes were allowed to proliferate for 5 days. As expected, we found that SNAR was not expressed in GFAP-positive cells but it strongly localized to neurons, as shown by colocalization with MAP2-positive cells (FIG. 5C).

### Example 8

### Conditional Expression of SNAR

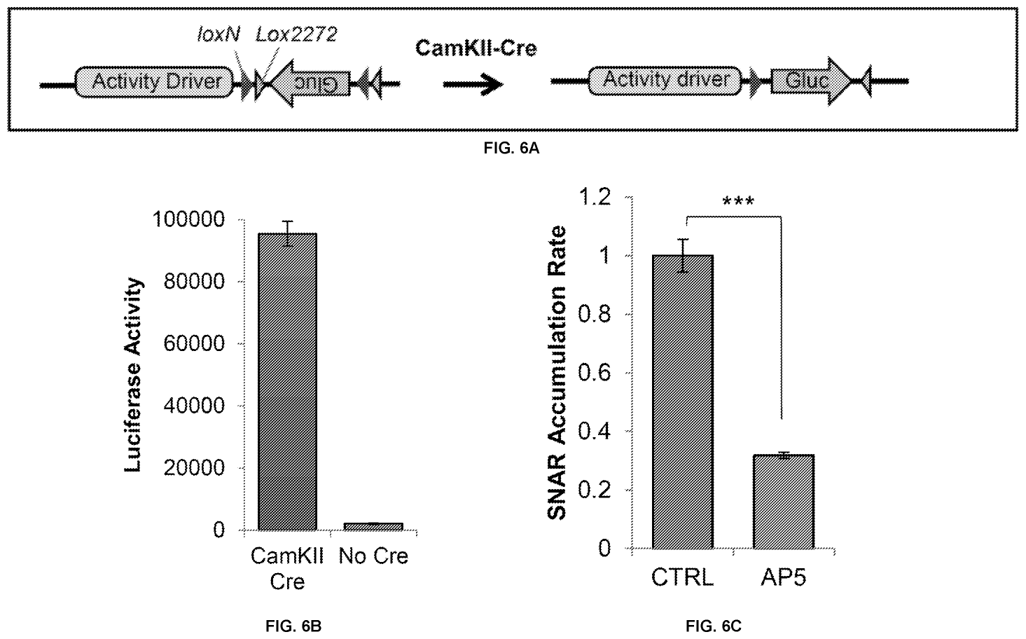

Having the reporter be expressed in a cell-type specific manner may be advantageous since a number of neurological disorders are caused by cell-type specific defects. The SNAR reporter may be a useful tool to study such disorders in vitro. Therefore, we inserted two lox sites (loxP and lox2272) flanking each luciferase sequence such that they would only be expressed in the presence of Cre recombinase. To demonstrate that we could combine the Cre system with the SNAR reporter, we transduced neurons with the floxed version of the reporter as well as CamKII:Cre (FIG. 6A). We found there was very little expression of the reporter in the absence of Cre recombinase (FIG. 6B). However, with Cre expression SNAR was robustly expressed, and upon treatment with the NMDAR blocker, APV, we observed a decrease in reporter accumulation similar to that of the non-specific reporter (FIG. 3A, FIG. 6C). Therefore, by using a cell-type specific promoter to drive expression of Cre, the SNAR reporter was expressed specifically in a subpopulation of neurons without affecting its activity.

### Example 9

### Discussion

Presented is a novel live cell assay to quantify the long-term changes in neuronal activity. The assay is simple, fully automatable, and easily adaptable for high throughput drug screens. Sensitivity and robustness of the SNAR reporter required only a small fraction of culture medium (a couple of microliters) and thus the neuronal activity of the same population of neurons can be measured multiple times with minimum perturbation of the culture conditions.

Compared to conventional assays, our assay provides several advantages in studying the development of neuronal activity in normal and disease conditions:

(1) By repeatedly monitoring reporter accumulation from live neurons, the effect of drug treatments on reporter activity is normalized to basal activity of the same neurons before the treatment, which serves as an internal control for culture conditions including infection rate, neuronal survival, healthiness, and maturation status. This will significantly lower variability and provide stronger confidence of drug-screening hits by paired statistical analysis.

(2) Due to a neuron's remarkable ability to maintain a range of neuronal activity in response to long-term changes in network activity, drugs that initially suppress the synaptic transmission may cause a compensatory increase in the synaptic receptors and intrinsic excitability. Our assay is designed for multiple time point analysis and is useful in distinguishing the acute vs long-term effects of pharmacological manipulations. Furthermore, kinetic analysis may reveal drug resistance and undesired side effects of the pharmacological manipulation developed over time.

(3) The assay is extremely simple and cost-effective. Quantitative luminescence is measured by collecting a small amount of medium and mixing it with the respective substrate, a procedure that can be fully automated.

(4) Efficiency of virus-mediated introduction of the reporter enables its expression in many types of neurons, including neurons derived from mutant animals or differentiated from disease-bearing human cells. By performing the dual reporter assay of activity-dependent and constitutive drivers, we can easily quantify the baseline activity and developmental profile of mutant neurons.

(5) Unlike MEAs, which cannot specify the neuronal activity of a subpopulation of neurons, our conditional reporter is able to monitor the neuronal activity of only a subpopulation of neurons, while maintaining the activity of other types of neurons intact.

(6) The conditional expression of the reporter is particularly useful to monitor the neuronal activity only in the mutated neurons when a mutation is introduced into post-mitotic neurons in a mosaic fashion by genetic engineering techniques such as CRISPR/HITI (Suzuki et al., 2016).

### Considerations for Drug Screen

(1) Drug Screening Targets: Synapse development and function are regulated at distinct steps, which include initial contact of neurites, formation of immature synapses, maturation, elimination, homeostatic regulation, and excitatory/inhibitory balancing (Clarke and Barres, 2013; Sudhof, 2017). Although our assay provides a temporal resolution by which stage-specific effects of genetic or pharmacological manipulations are revealed, it provides a limited mechanistic insight. Our reporter is designed to screen the entire pathway. To distinguish whether a specific manipulation alters early developmental process of synaptogenesis or directly modulates synaptic transmission per se, independent assays including immunostainings and electrophysiological analyses need to be performed. Synapse development and function can be affected by non-specific effects such as impaired energy metabolism and viability of neighboring neurons. Therefore, the effects of each hit on the control reporter and cell viability need to be independently validated.

(2) Due to a neuron's remarkable ability to adapt to long-term changes in network activity, drugs that initially suppress synaptic transmission may have unexpected long-term effects due to a compensatory increase in synaptic receptors or intrinsic excitability. Because the SNAR assay may be used for multiple time-point measurements, it is suitable to identify and distinguish acute and long-term effects of pharmacological manipulations. Furthermore, kinetic analyses may reveal drug resistance or other undesired side effects developed over time.

(3) Lagging time: Lagging time may be considered when experiment planning. Due to the on-going release of pre-synthesized proteins in the secretory pathway and newly synthesized proteins from the pre-transcribed mRNA, detecting a significant reduction below the baseline can be challenging within 14-16 hours after the beginning of treatment. The reporter could be improved by using a destabilized mRNA and protein to shorten the lagging time and thus improve its temporal resolution.

(4) Spike-like activation of the reporter activity upon stimulation. Stimulation of neuronal activity after the washout of inhibitors rapidly induces reporter activity within 30 minutes. It is notable that after the 30 minutes, the promoter activity was reduced basal rate (FIG. 1D), which reveals the transient, spike-like activity of the IEG expression. The bi-phasic response of the promoter activity may be due to the transient depletion of the pre-initiation complex. Thus short term monitoring of the SNAR activity is recommended to identify an activator of the neuronal activity.

(5) Excitation and inhibitory (Ell) balance: E/I balance is tightly controlled and is often impaired in disease conditions. It is important to distinguish whether overall changes in the network activity are caused by a direct effect on excitatory neurons or the opposite effect on the inhibitory neurons. Moreover, if a drug inhibits both excitatory and inhibitory inputs to the same degree, E/I balance will be maintained and thus may not be detected by the assay causing false negative results. Conditional expression of the reporter only in excitatory or inhibitory neurons is required to reveal the role of cell-type specific effect of a drug treatment in the network activity and to reduce the false negative rate.

Modulation of neuronal activity in specific types of neurons and mutant neurons. Many studies to identify the modulator of synapse development and function have focused on excitatory neurons. However, recent genetic studies of human patient show that inhibitory neurons play key roles in neurodevelopmental and neuropsychiatric diseases. Whether the development and function of the synapses on inhibitory neurons follows the same program as excitatory neurons is largely unknown. Our genetic reporter allows to isolate the changes in specific types of neurons and revealed that epileptic drugs and astrocyte-derived synaptogenic factors differently affect the neuronal activities of excitatory neurons vs inhibitory neurons, indicating that the synapse development of inhibitory neurons is regulated via a different mechanism. Thus, selective drug screens should be employed to identify drugs that specifically modulate inhibitory neurons. In addition to inhibitory neurons, our assay will be useful to isolate the neuronal activity in even sub-population of inhibitory neurons and other specific types of neurons including dopaminergic and serotonergic neurons in combination with specific Cre drivers. Moreover, this assay is useful to monitor the development profile of mutant neurons derived from the patient.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A Secreted Neuronal Activity Reporter (SNAR) construct comprising: four tandem repeats of a core domain of the Synaptic Activity Response Element (SARE) of Arc/Arg3.1; a polynucleotide comprising the Arc minimal promoter; and a polynucleotide encoding a first secreted reporter protein.

Clause 2. The SNAR construct of clause 1, wherein the core domain of the SARE of Arc/Arg3.1 comprises a polynucleotide of SEQ ID NO: 2.

Clause 3. The SNAR construct of clause 1 or 2, wherein the Arc minimal promoter comprises a polynucleotide of SEQ ID NO: 3.

Clause 4. The SNAR construct of any one of clauses 1-3, wherein the first secreted reporter protein emits a light signal upon contact with a substrate.

Clause 5. The SNAR construct of clause 4, wherein the substrate comprises coelenterazine.

Clause 6. The SNAR construct of any one of clauses 1-5, wherein the first secreted reporter protein comprises Gaussia luciferase.

Clause 7. The SNAR construct of clause 6, wherein the Gaussia luciferase comprises a polypeptide of SEQ ID NO: 7.

Clause 8. The SNAR construct of any one of clauses 1-7, further comprising a loxP site upstream of the polynucleotide encoding a first secreted reporter protein, and a lox2272 site downstream of the polynucleotide encoding a first secreted reporter protein.

Clause 9. The SNAR construct of any one of clauses 1-7, further comprising a loxP site downstream of the polynucleotide encoding a first secreted reporter protein, and a lox2272 site upstream of the polynucleotide encoding a first secreted reporter protein.

Clause 10. The SNAR construct of clause 8 or 9, wherein the loxP site comprises a polynucleotide of SEQ ID NO: 4, and wherein the lox2272 site comprises a polynucleotide of SEQ ID NO: 5.

Clause 11. A neuronal cell activity reporter system comprising: (a) the SNAR construct of any one of clauses 1-10; and (b) a control construct comprising: a polynucleotide comprising a constitutive promoter; and a polynucleotide encoding a second secreted reporter protein.

Clause 12. A method of monitoring neuronal activity in a test cell, the method comprising: administering to the test cell the SNAR construct of any one of clauses 1-10; contacting the first secreted reporter protein with a substrate, wherein the substrate reacts with the first secreted reporter protein to generate a first signal (CTZₛₐₘₚₗₑ); measuring the first signal; and determining the neuronal activity in the test cell based on the first signal.

Clause 13. The method of clause 12, wherein the substrate comprises coelenterazine.

Clause 14. The method of any one of clauses 12-13, wherein the first secreted reporter protein is exported out of the test cell to a culture medium.

Clause 15. The method of clause 14, wherein the first secreted reporter protein is contacted with the substrate by adding the substrate to a sample of the culture medium.

Clause 16. The method of any one of clauses 12-15, wherein the first signal is measured at two different time points, and wherein the neuronal activity in the test cell at the two different time points are compared.

Clause 17. The method of any one of clauses 12-15, wherein the neuronal activity in the test cell is monitored by measuring the first signal at a plurality of different time points.

Clause 18. A method of monitoring neuronal activity in a test cell, the method comprising: (a) administering to the test cell the SNAR construct of any one of clauses 1-10, and a control construct, the control construct comprising: a polynucleotide comprising a constitutive promoter; and a polynucleotide encoding a second secreted reporter protein; (b) contacting the first secreted reporter protein and the second secreted reporter protein in the test cell with a first substrate, wherein the first substrate reacts with the first secreted reporter protein and the second secreted reporter protein to generate a first signal (CTZₛₐₘₚₗₑ); (c) measuring the first signal; (d) contacting the first secreted reporter protein and the second secreted reporter protein in the test cell with a second substrate, wherein the second substrate reacts with the second secreted reporter protein to generate a second signal (FMZₛₐₘₚₗₑ); (e) measuring the second signal; (f) administering to a control cell the control construct of step (a); (g) contacting the second secreted reporter protein in the control cell with the first substrate, wherein the first substrate reacts with the second secreted reporter protein to generate a third signal (CTZ_{sNluc}); (h) measuring the third signal; (i) contacting the second secreted reporter protein in the control cell with the second substrate, wherein the second substrate reacts with the second secreted reporter protein to generate a fourth signal (FMZ_{sNluc}); (j) measuring the fourth signal; (k) determining a control ratio by dividing the third signal by the fourth signal (CTZ_{sNluc}/FMZ_{sNluc}); and (l) determining the neuronal activity in the test cell based on the contribution of the first secreted reporter protein to the first signal with the control ratio.

Clause 19. The method of clause 18, wherein the contribution of the first secreted reporter protein to the first signal is calculated by subtracting from the first signal the product of the control ratio and the second signal (first signal−[(third signal/fourth signal]×second signal]=CTZₛₐₘₚₗₑ−[(CTZ_{sNluc}/FMZ_{cNluc})×FMZₛₐₘₚₗₑ]).

Clause 20. The neuronal cell activity reporter system of clause 11 or the method of any one of clauses 18-19, wherein the constitutive promoter comprises a human PGK promoter.

Clause 21. The neuronal cell activity reporter system or the method of clause 20, wherein the human PGK promoter comprises a polynucleotide of SEQ ID NO: 6.

Clause 22. The neuronal cell activity reporter system of clause 11 or the method of any one of clauses 18-21, wherein the second secreted reporter protein emits a signal upon contact with a substrate, the signal being distinct from the signal emitted by the first secreted reporter protein upon contact with a substrate.

Clause 23. The neuronal cell activity reporter system or the method of clause 22, wherein the second secreted reporter protein emits a signal upon contact with furimazine, coelenterazine, or a combination thereof.

Clause 24. The method of any one of clauses 18-23, wherein the first substrate comprises coelenterazine.

Clause 25. The method of any one of clauses 18-24, wherein the second substrate comprises furimazine.

Clause 26. The neuronal cell activity reporter system of clause 11 or the method of any one of clauses 18-25, wherein the second secreted reporter protein comprises a nanoluciferase comprising an N-terminal secretion signal peptide.

Clause 27. The neuronal cell activity reporter system or the method of clause 26, wherein the nanoluciferase comprising an N-terminal secretion signal peptide comprises a polypeptide of SEQ ID NO: 9.

Clause 28. The neuronal cell activity reporter system of clause 11 or the method of any one of clauses 18-27, wherein the control construct further comprises a loxP site upstream of the polynucleotide encoding a second secreted reporter protein, and a lox2272 site downstream of the polynucleotide encoding a second secreted reporter protein.

Clause 29. The neuronal cell activity reporter system of clause 11 or the method of any one of clauses 18-28, wherein the control construct further comprises a loxP site downstream of the polynucleotide encoding a second secreted reporter protein, and a lox2272 site upstream of the polynucleotide encoding a second secreted reporter protein.

Clause 30. The neuronal cell activity reporter system or the method of clause 28 or 29, wherein the loxP site comprises a polynucleotide sequence of SEQ ID NO: 4, and wherein the lox2272 site comprises a polynucleotide sequence of SEQ ID NO: 5.

Clause 31. The method of any one of clauses 18-30, wherein the first secreted reporter protein and the second secreted reporter protein are exported out of the test cell to a culture medium.

Clause 32. The method of clause 31, wherein the first secreted reporter protein and the second secreted reporter protein are contacted with the first substrate by adding the first substrate to a sample of the culture medium.

Clause 33. The method of clause 31, wherein the first secreted reporter protein and the second secreted reporter protein are contacted with the second substrate by adding the second substrate to a sample of the culture medium.

Clause 34. The method of any one of clauses 18-33, wherein the first signal and the second signal are measured at two different time points, and wherein the neuronal activity in the test cell at the two different time points are compared.

Clause 35. The method of any one of clauses 18-34, wherein the neuronal activity in the test cell is monitored by measuring the first signal and the second signal at a plurality of different time points.

Clause 36. The method of any one of clauses 12-35, wherein the method further comprises contacting the test cell with a Cre recombinase.

Clause 37. The method of any one of clauses 12-36, wherein the test cell is a live cell.

Clause 38. The method of any one of clauses 12-37, wherein the method further comprises contacting the test cell with a modulator of synaptic signaling.

Clause 39. The SNAR construct of any one of clauses 1-10, or the neuronal cell activity reporter system of any one of clauses 11, 20-23, and 26-30, or the method of any one of clauses 12-38, wherein the SNAR construct is an adeno-associated virus (AAV) or a lentivirus.

SEQUENCES
Polynucleotide sequence of the synaptic activity response element (SARE) of
Arc/Arg3.1 (104 nt)
SEQ ID NO: 1
agcgcacagagccttcctgcgtggggaagctccttgctgcgtcatggctcagctattctcag
cctctctccttttatggtgccggaagcaggcaggctgctgct
Polynucleotide sequence of the core domain of the SARE of Arc/Arg3.1 (85 nt)
SEQ ID NO: 2
cctgcgtggggaagctccttgctgcgtcatggctcagctattctcagcctctctccttttat
ggtgccggaagcaggcaggctgc
Polynucleotide sequence of the Arc minimal promoter (414 nt)
SEQ ID NO: 3
cagagcacattagtcactcggggctgtgaaggggcgggtccttgagggcacccacgggaggggagcgagtaggc
gcggaaggcggggcctgcggcaggagagggcgcgggcgggctctggcgcggagcctgggcgccgccaatggg
agccagggctccacgagctgccgcccacgggccccgcgcagcataaatagccgctggtggcggtttcggtgcaga
gctcaagcgagttctcccgcagccgcagtctctgggcctctctagcttcagcggcgacgagcctgccacactcgctaa
gctcctccggcaccgcacacctgccactgccgctgcagccgccggctctgctcccttccggcttctgcctcagaggag
ttcttagcctgttcggagccgcagcaccgacgaccag
Polynucleotide sequence of the loxP site
SEQ ID NO: 4
ataacttcgtatagcatacattatacgaagttat
Polynucleotide sequence of the lox2272 site
SEQ ID NO: 5
ataacttcgtataggatactttatacgaagttat
Polynucleotide sequence of the human PGK promoter (511 nt)
SEQ ID NO: 6
ggggttggggttgcgccttttccaaggcagccctgggtttgcgcagggacgcggctgctctgggcgtggttccgggaa
acgcagcggcgccgaccctgggtctcgcacattcttcacgtccgttcgcagcgtcacccggatcttcgccgctaccctt
gtgggccccccggcgacgcttcctgctccgcccctaagtcgggaaggttccttgcggttcgcggcgtgccggacgtg
acaaacggaagccgcacgtctcactagtaccctcgcagacggacagcgccagggagcaatggcagcgcgccga
ccgcgatgggctgtggccaatagcggctgctcagcagggcgcgccgagagcagcggccgggaaggggcggtgc
gggaggcggggtgtggggcggtagtgtgggccctgttcctgcccgcgcggtgttccgcattctgcaagcctccggag
cgcacgtcggcagtcggctccctcgttgaccgaatcaccgacctctctccccag
Gaussia luciferase polypeptide
SEQ ID NO: 7
MGVKVLFALICIAVAEAKPTENNEDFNIVAVASNFATTDLDADRGKLPGKKLPLEVLK
EMEANARKAGCTRGCLICLSHIKCTPKMKKFIPGRCHTYEGDKESAQGGIGEAIVDI
PEIPGFKDLEPMEQFIAQVDLCVDCTTGCLKGLANVQCSDLLKKWLPQRCATFASKI
QGQVDKIKGAGGD
Polynucleotide sequence encoding Gaussia luciferase (558 nt)
SEQ ID NO: 8
atgggagtcaaagttctgtttgccctgatctgcatcgctgtggccgaggccaagcccaccgagaacaacgaagactt
caacatcgtggccgtggccagcaacttcgcgaccacggatctcgatgctgaccgcgggaagttgcccggcaagaa
gctgccgctggaggtgctcaaagagatggaagccaatgcccggaaagctggctgcaccaggggctgtctgatctgc
ctgtcccacatcaagtgcacgcccaagatgaagaagttcatcccaggacgctgccacacctacgaaggcgacaaa
gagtccgcacagggcggcataggcgaggcgatcgtcgacattcctgagattcctgggttcaaggacttggagcccat
ggagcagttcatcgcacaggtcgatctgtgtgtggactgcacaactggctgcctcaaagggcttgccaacgtgcagtg
ttctgacctgctcaagaagtggctgccgcaacgctgtgcgacctttgccagcaagatccagggccaggtggacaag
atcaagggggccggtggtgactaa
Second secreted reporter protein secreted nanoluciferase (sNluc)
SEQ ID NO: 9
VFTLEDFVGDWRQTAGYNLDQVLEQGGVSSLFQNLGVSVTPIQRIVLSGENGLKIDI
HVIIPYEGLSGDQMGQIEKIFKVVYPVDDHHFKVILHYGTLVIDGVTPNMIDYFGRPY
EGIAVFDGKKITVTGTLWNGNKIIDERLINPDGSLLFRVTINGVTGWRLCERILA
Polynucleotide sequence encoding secreted nanoluciferase (618 nt)
SEQ ID NO: 10
atggagacagacacactcctgctatgggtactgctgctctgggttccaggttccactggtgacactagttatccatatgat
gttccagattatgctggtggatcagtcttcacactcgaagatttcgttggggactggcgacagacagccggctacaacc
tggaccaagtccttgaacagggaggtgtgtccagtttgtttcagaatctcggggtgtccgtaactccgatccaaaggatt
gtcctgagcggtgaaaatgggctgaagatcgacatccatgtcatcatcccgtatgaaggtctgagcggcgaccaaat
gggccagatcgaaaaaatttttaaggtggtgtaccctgtggatgatcatcactttaaggtgatcctgcactatggcacac
tggtaatcgacggggttacgccgaacatgatcgactatttcggacggccgtatgaaggcatcgccgtgttcgacggca
aaaagatcactgtaacagggaccctgtggaacggcaacaaaattatcgacgagcgcctgatcaaccccgacggct
ccctgctgttccgagtaaccatcaacggagtgaccggctggcggctgtgcgaacgcattctggcgtaa
Secretion signal peptide
SEQ ID NO: 11
METDTLLLWVLLLWVPGSTGD
Polynucleotide sequence of the SNAR construct
SEQ ID NO: 12
cctgcgtggggaagctccttgctgcgtcatggctcagctattctcagcctctctccttttat
ggtgccggaagcaggcaggctgccgcgtagcctgcctgcgtggggaagctccttgctgcgtc
atggctcagctattctcagcctctctccttttatggtgccggaagcaggcaggctgccgcgt
agcctgcctgcgtggggaagctccttgctgcgtcatggctcagctattctcagcctctctcc
ttttatggtgccggaagcaggcaggctgcagccttcctgcgtggggaagctccttgctgcgt
catggctcagctattctcagcctctctccttttatggtgccggaagcaggcaggctgcagat
ctcgcgcagcagagcacattagtcactcggggctgtgaaggggcgggtccttgagggcaccc
acgggaggggagcgagtaggcgcggaaggcggggcctgcggcaggagagggcgcgggcgggc
tctggcgcggagcctgggcgccgccaatgggagccagggctccacgagctgccgcccacggg
ccccgcgcagcataaatagccgctggtggcggtttcggtgcagagctcaagcgagttctccc
gcagccgcagtctctgggcctctctagcttcagcggcgacgagcctgccacactcgctaagc
tcctccggcaccgcacacctgccactgccgctgcagccgccggctctgctcccttccggctt
ctgcctcagaggagttcttagcctgttcggagccgcagcaccgacgaccagaagcttggtac
cgagctcggatccagccaccatgggagtcaaagttctgtttgccctgatctgcatcgctgtg
gccgaggccaagcccaccgagaacaacgaagacttcaacatcgtggccgtggccagcaactt
cgcgaccacggatctcgatgctgaccgcgggaagttgcccggcaagaagctgccgctggagg
tgctcaaagagatggaagccaatgcccggaaagctggctgcaccaggggctgtctgatctgc
ctgtcccacatcaagtgcacgcccaagatgaagaagttcatcccaggacgctgccacaccta
cgaaggcgacaaagagtccgcacagggcggcataggcgaggcgatcgtcgacattcctgaga
ttcctgggttcaaggacttggagcccatggagcagttcatcgcacaggtcgatctgtgtgtg
gactgcacaactggctgcctcaaagggcttgccaacgtgcagtgttctgacctgctcaagaa
gtggctgccgcaacgctgtgcgacctttgccagcaagatccagggccaggtggacaagatca
agggggccggtggtgactaa
Polynucleotide sequence of a control construct
SEQ ID NO: 13
ggggttggggttgcgccttttccaaggcagccctgggtttgcgcagggacgcggctgctctg
ggcgtggttccgggaaacgcagcggcgccgaccctgggtctcgcacattcttcacgtccgtt
cgcagcgtcacccggatcttcgccgctacccttgtgggccccccggcgacgcttcctgctcc
gcccctaagtcgggaaggttccttgcggttcgcggcgtgccggacgtgacaaacggaagccg
cacgtctcactagtaccctcgcagacggacagcgccagggagcaatggcagcgcgccgaccg
cgatgggctgtggccaatagcggctgctcagcagggcgcgccgagagcagcggccgggaagg
ggcggtgcgggaggcggggtgtggggcggtagtgtgggccctgttcctgcccgcgcggtgtt
ccgcattctgcaagcctccggagcgcacgtcggcagtcggctccctcgttgaccgaatcacc
gacctctctccccagggggatccaccggttcgtcgactagtccagtgtggtggaattcgcca
ccatggagacagacacactcctgctatgggtactgctgctctgggttccaggttccactggt
gacactagttatccatatgatgttccagattatgctggtggatcagtcttcacactcgaaga
tttcgttggggactggcgacagacagccggctacaacctggaccaagtccttgaacagggag
gtgtgtccagtttgtttcagaatctcggggtgtccgtaactccgatccaaaggattgtcctg
agcggtgaaaatgggctgaagatcgacatccatgtcatcatcccgtatgaaggtctgagcgg
cgaccaaatgggccagatcgaaaaaatttttaaggtggtgtaccctgtggatgatcatcact
ttaaggtgatcctgcactatggcacactggtaatcgacggggttacgccgaacatgatcgac
tatttcggacggccgtatgaaggcatcgccgtgttcgacggcaaaaagatcactgtaacagg
gaccctgtggaacggcaacaaaattatcgacgagcgcctgatcaaccccgacggctccctgc
tgttccgagtaaccatcaacggagtgaccggctggcggctgtgcgaacgcattctggcgtaa

1. A Secreted Neuronal Activity Reporter (SNAR) construct comprising:
four tandem repeats of a core domain of the Synaptic Activity Response Element (SARE) of Arc/Arg3.1;
a polynucleotide comprising the Arc minimal promoter; and
a polynucleotide encoding a first secreted reporter protein.

2. The SNAR construct of claim 1, wherein the core domain of the SARE of Arc/Arg3.1 comprises a polynucleotide of SEQ ID NO: 2.

3. The SNAR construct of claim 1, wherein the Arc minimal promoter comprises a polynucleotide of SEQ ID NO: 3.

4. The SNAR construct of claim 1, wherein the first secreted reporter protein emits a light signal upon contact with a substrate.

5. The SNAR construct of claim 4, wherein the substrate comprises coelenterazine.

6. The SNAR construct of claim 1, wherein the first secreted reporter protein comprises Gaussia luciferase.

7. The SNAR construct of claim 6, wherein the Gaussia luciferase comprises a polypeptide of SEQ ID NO: 7.

8. The SNAR construct of claim 1, further comprising a loxP site upstream of the polynucleotide encoding a first secreted reporter protein, and a lox2272 site downstream of the polynucleotide encoding a first secreted reporter protein.

9. The SNAR construct of claim 1, further comprising a loxP site downstream of the polynucleotide encoding a first secreted reporter protein, and a lox2272 site upstream of the polynucleotide encoding a first secreted reporter protein.

10. The SNAR construct of claim 8, wherein the loxP site comprises a polynucleotide of SEQ ID NO: 4, and wherein the lox2272 site comprises a polynucleotide of SEQ ID NO: 5.

11. A neuronal cell activity reporter system comprising:
(a) the SNAR construct of claim 1; and
(b) a control construct comprising:
a polynucleotide comprising a constitutive promoter; and
a polynucleotide encoding a second secreted reporter protein.

12. A method of monitoring neuronal activity in a test cell, the method comprising:
administering to the test cell the SNAR construct of claim 1;
contacting the first secreted reporter protein with a substrate, wherein the substrate reacts with the first secreted reporter protein to generate a first signal (CTZₛₐₘₚₗₑ);
measuring the first signal; and
determining the neuronal activity in the test cell based on the first signal.

13. The method of claim 12, wherein the substrate comprises coelenterazine.

14. The method of claim 12, wherein the first secreted reporter protein is exported out of the test cell to a culture medium.

15. The method of claim 14, wherein the first secreted reporter protein is contacted with the substrate by adding the substrate to a sample of the culture medium.

16. The method of claim 12, wherein the first signal is measured at two different time points, and wherein the neuronal activity in the test cell at the two different time points are compared.

17. The method of claim 12, wherein the neuronal activity in the test cell is monitored by measuring the first signal at a plurality of different time points.

18. A method of monitoring neuronal activity in a test cell, the method comprising:
(a) administering to the test cell the SNAR construct of claim 1, and a control construct, the control construct comprising:
a polynucleotide comprising a constitutive promoter; and
a polynucleotide encoding a second secreted reporter protein;
(b) contacting the first secreted reporter protein and the second secreted reporter protein in the test cell with a first substrate, wherein the first substrate reacts with the first secreted reporter protein and the second secreted reporter protein to generate a first signal (CTZₛₐₘₚₗₑ);
(c) measuring the first signal;
(d) contacting the first secreted reporter protein and the second secreted reporter protein in the test cell with a second substrate, wherein the second substrate reacts with the second secreted reporter protein to generate a second signal (FMZₛₐₘₚₗₑ);
(e) measuring the second signal;
(f) administering to a control cell the control construct of step (a);
(g) contacting the second secreted reporter protein in the control cell with the first substrate, wherein the first substrate reacts with the second secreted reporter protein to generate a third signal (CTZ_{sNluc});
(h) measuring the third signal;
(i) contacting the second secreted reporter protein in the control cell with the second substrate, wherein the second substrate reacts with the second secreted reporter protein to generate a fourth signal (FMZ_{sNluc});
(j) measuring the fourth signal;
(k) determining a control ratio by dividing the third signal by the fourth signal (CTZ_{sNluc}/FMZ_{sNluc}); and
(l) determining the neuronal activity in the test cell based on the contribution of the first secreted reporter protein to the first signal with the control ratio.

19. The method of claim 18, wherein the contribution of the first secreted reporter protein to the first signal is calculated by subtracting from the first signal the product of the control ratio and the second signal (first signal−[(third signal/fourth signal]×second signal]=CTZₛₐₘₚₗₑ−[(CTZ_{sNluc}/FMZ_{cNluc})×FMZₛₐₘₚₗₑ]).

20. The neuronal cell activity reporter system of claim 11, wherein the constitutive promoter comprises a human PGK promoter.

21. The neuronal cell activity reporter system of claim 20, wherein the human PGK promoter comprises a polynucleotide of SEQ ID NO: 6.

22. The neuronal cell activity reporter system of claim 11, wherein the second secreted reporter protein emits a signal upon contact with a substrate, the signal being distinct from the signal emitted by the first secreted reporter protein upon contact with a substrate.

23. The neuronal cell activity reporter system of claim 22, wherein the second secreted reporter protein emits a signal upon contact with furimazine, coelenterazine, or a combination thereof.

24. The method of claim 18, wherein the first substrate comprises coelenterazine.

25. The method of claim 18, wherein the second substrate comprises furimazine.

26. The neuronal cell activity reporter system of claim 11, wherein the second secreted reporter protein comprises a nanoluciferase comprising an N-terminal secretion signal peptide.

27. The neuronal cell activity reporter system of claim 26, wherein the nanoluciferase comprising an N-terminal secretion signal peptide comprises a polypeptide of SEQ ID NO: 9.

28. The neuronal cell activity reporter system of claim 11, wherein the control construct further comprises a loxP site upstream of the polynucleotide encoding a second secreted reporter protein, and a lox2272 site downstream of the polynucleotide encoding a second secreted reporter protein.

29. The neuronal cell activity reporter system of claim 11, wherein the control construct further comprises a loxP site downstream of the polynucleotide encoding a second secreted reporter protein, and a lox2272 site upstream of the polynucleotide encoding a second secreted reporter protein.

30. The neuronal cell activity reporter system of claim 28, wherein the loxP site comprises a polynucleotide sequence of SEQ ID NO: 4, and wherein the lox2272 site comprises a polynucleotide sequence of SEQ ID NO: 5.

31. The method of claim 18, wherein the first secreted reporter protein and the second secreted reporter protein are exported out of the test cell to a culture medium.

32. The method of claim 31, wherein the first secreted reporter protein and the second secreted reporter protein are contacted with the first substrate by adding the first substrate to a sample of the culture medium.

33. The method of claim 31, wherein the first secreted reporter protein and the second secreted reporter protein are contacted with the second substrate by adding the second substrate to a sample of the culture medium.

34. The method of claim 18, wherein the first signal and the second signal are measured at two different time points, and wherein the neuronal activity in the test cell at the two different time points are compared.

35. The method of claim 18, wherein the neuronal activity in the test cell is monitored by measuring the first signal and the second signal at a plurality of different time points.

36. The method of claim 12, wherein the method further comprises contacting the test cell with a Cre recombinase.

37. The method of claim 12, wherein the test cell is a live cell.

38. The method of claim 12, wherein the method further comprises contacting the test cell with a modulator of synaptic signaling.

39. The SNAR construct of claim 1, wherein the SNAR construct is an adeno-associated virus (AAV) or a lentivirus.


SEQUENCE LISTING (13 sequences)

SEQ ID NO: 1 - DNA, 104 bp, Artificial Sequence
  Synthetic
agcgcacagagccttcctgcgtggggaagctccttgctgcgtcatggctcagctattctcagcctctctccttttatggtgccggaagcaggcaggctgctgct

SEQ ID NO: 2 - DNA, 85 bp, Artificial Sequence
  Synthetic
cctgcgtggggaagctccttgctgcgtcatggctcagctattctcagcctctctccttttatggtgccggaagcaggcaggctgc

SEQ ID NO: 3 - DNA, 414 bp, Artificial Sequence
  Synthetic
cagagcacattagtcactcggggctgtgaaggggcgggtccttgagggcacccacgggaggggagcgagtaggcgcggaaggcggggcctgcggcaggagagggcgcgggcgggctctggcgcggagcctgggcgccgccaatgggagccagggctccacgagctgccgcccacgggccccgcgcagcataaatagccgctggtggcggtttcggtgcagagctcaagcgagttctcccgcagccgcagtctctgggcctctctagcttcagcggcgacgagcctgccacactcgctaagctcctccggcaccgcacacctgccactgccgctgcagccgccggctctgctcccttccggcttctgcctcagaggagttcttagcctgttcggagccgcagcaccgacgaccag

SEQ ID NO: 4 - DNA, 34 bp, Artificial Sequence
  Synthetic
ataacttcgtatagcatacattatacgaagttat

SEQ ID NO: 5 - DNA, 34 bp, Artificial Sequence
  Synthetic
ataacttcgtataggatactttatacgaagttat

SEQ ID NO: 6 - DNA, 511 bp, Artificial Sequence
  Synthetic
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 7 - protein, 185 aa, Artificial Sequence
  Synthetic
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 8 - DNA, 558 bp, Artificial Sequence
  Synthetic
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 9 - protein, 170 aa, Artificial Sequence
  Synthetic
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 10 - DNA, 618 bp, Artificial Sequence
  Synthetic
atggagacagacacactcctgctatgggtactgctgctctgggttccaggttccactggtgacactagttatccatatgatgttccagattatgctggtggatcagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 11 - protein, 21 aa, Artificial Sequence
  Synthetic
METDTLLLWVLLLWVPGSTGD

SEQ ID NO: 12 - DNA, 1384 bp, Artificial Sequence
  Synthetic
cctgcgtggggaagctccttgctgcgtcatggctcagctattctcagcctctctccttttatggtgccggaagcaggcaggctgccgcgtagcctgcctgcgtggggaagctccttgctgcgtcatggctcagctattctcagcctctctccttttatggtgccggaagcaggcaggctgccgcgtagcctgcctgcgtggggaagctccttgctgcgtcatggctcagctattctcagcctctctccttttatggtgccggaagcaggcaggctgcagccttcctgcgtggggaagctccttgctgcgtcatggctcagctattctcagcctctctccttttatggtgccggaagcaggcaggctgcagatctcgcgcagcagagcacattagtcactcggggctgtgaaggggcgggtccttgagggcacccacgggaggggagcgagtaggcgcggaaggcggggcctgcggcaggagagggcgcgggcgggctctggcgcggagcctgggcgccgccaatgggagccagggctccacgagctgccgcccacgggccccgcgcagcataaatagccgctggtggcggtttcggtgcagagctcaagcgagttctcccgcagccgcagtctctgggcctctctagcttcagcggcgacgagcctgccacactcgctaagctcctccggcaccgcacacctgccactgccgctgcagccgccggctctgctcccttccggcttctgcctcagaggagttcttagcctgttcggagccgcagcaccgacgaccagaagcttggtaccgagctcggatccagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 13 - DNA, 1178 bp, Artificial Sequence
  Synthetic
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggttcgtcgactagtccagtgtggtggaattcgccaccatggagacagacacactcctgctatgggtactgctgctctgggttccaggttccactggtgacactagttatccatatgatgttccagattatgctggtggatcagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa